(19) United States
(12) Patent Application Publication
(10) Pub. No.: US 2019/0142546 A1
(43) Pub. Date: May 16, 2019

(54) MEDICAL DEVICE FLUSHING SYSTEMS AND METHODS

(71) Applicant: Kardium Inc., Bumaby (CA)

(72) Inventors: Ian GARBEN, Burnaby (CA); Calvin Dane CUMMINGS, Surrey (CA); John Andrew FUNK, Delta (CA)

(21) Appl. No.: 16/151,740

(22) Filed: Oct. 4, 2018

Related U.S. Application Data
(60) Provisional application No. 62/584,372, filed on Nov. 10, 2017.

(51) Int. Cl. A61B 90/70; A61B 18/14

(52) U.S. Cl. CPC A61B 90/70; A61B 2018/00267; A61B 18/1492

(57) ABSTRACT

A medical device flushing apparatus may include an enclosure and at least one fluid-providing channel. The enclosure may include an interior surface defining at least part of a flushing cavity configured to receive a structure of a medical device. The at least one fluid-providing channel may interrupt the interior surface of the enclosure, the at least one fluid-providing channel configured to provide fluid to the flushing cavity. Each of the at least one fluid-providing channel may point, in a fluid-providing direction, toward an interior of the flushing cavity, each fluid-providing direction pointing away from a centroid of the flushing cavity.

### CROSS-REFERENCE TO RELATED APPLICATION

This application claims the benefit of U.S. Provisional Application No. 62/584,372, filed Nov. 10, 2017, the entire disclosure of which is hereby incorporated herein by reference.

### TECHNICAL FIELD

Aspects of this disclosure generally are related to apparatuses and methods for flushing a medical device system, such as a catheter device system, of an undesired fluid. According to some embodiments, apparatuses and methods are disclosed for flushing an undesired fluid from a controllable manipulable portion of a catheter device system.

### BACKGROUND

Cardiac surgery was initially undertaken using highly invasive open procedures. A sternotomy, which is a type of incision in the center of the chest that separates the sternum, was typically employed to allow access to the heart. In the past several decades, more and more cardiac operations are performed using intravascular or percutaneous techniques, where access to inner organs or other tissue is gained via a catheter.

Intravascular or percutaneous surgeries benefit patients by reducing surgery risk, complications, and recovery time. However, the use of intravascular or percutaneous technologies also raises some particular challenges. Medical devices used in intravascular or percutaneous surgery need to be deployed via catheter systems which significantly increase the complexity of the device structure. As well, doctors do not have direct visual contact with the medical devices once the devices are positioned within the body.

One example of where intravascular or percutaneous medical techniques have been employed is in the treatment of a heart disorder called atrial fibrillation. Atrial fibrillation is a disorder in which spurious electrical signals cause an irregular heartbeat. Atrial fibrillation has been treated with open heart methods using a technique known as the “Cox-Maze procedure”. During various procedures, health care providers create specific patterns of lesions in the left or right atria to block various paths taken by the spurious electrical signals. Such lesions were originally created using incisions, but are now typically created by ablating the tissue with various techniques including radio-frequency (RF) energy, microwave energy, laser energy, and cryogenic techniques.

Preparation of catheter device systems for subsequent delivery through a bodily opening leading to a bodily cavity (e.g., as required by some percutaneous or intravascular procedures) may require that various fluids (e.g., air) be purged or otherwise removed from portions of the systems prior to insertion into the body. Failure to do so may allow for a transfer of at least some of the fluids to within the body which may in turn result in various undesired outcomes (e.g., the formation of various air embolisms). Various catheter device systems employ various features that can act as fluid traps from which undesired fluid may be difficult to remove therefrom. For example, various channels produced by various elements of the catheter member or medical instrument (e.g., converging elongated elements of basket-type catheter devices or converging members of various implants) may act as fluid traps. Even various materials that may be employed by various catheter device systems may make it difficult to remove undesired fluid. For example, polytetrafluoroethylene (PTFE) is typically employed by various catheter device systems because of its relatively low friction characteristics. However, polytetrafluoroethylene is an example of a material that essentially is hydrophobic in nature, and, thus, can restrict removal of fluid bubbles on a surface thereof when a water-based liquid (e.g., saline) is employed to flush or otherwise remove the fluid bubbles. That is, the flow of the flushing liquid may be insufficient to flush bubbles accumulated on a surface of the lumen, especially when that surface has hydrophobic characteristics which tend to repel the liquid, thereby reducing the flushing ability of the liquid.

Accordingly, a need in the art exists for systems and methods having improved capabilities for the removal of undesired fluid from medical device systems, such as catheter device systems.

### SUMMARY

At least the above-discussed need is addressed and technical solutions are achieved in the art by various embodiments of the present invention. In some embodiments, a medical device flushing apparatus may be summarized as including an enclosure including an interior surface defining at least part of a flushing cavity configured to receive a structure of a medical device, and at least one fluid-providing channel interrupting the interior surface of the enclosure, the at least one fluid-providing channel configured to provide fluid to the flushing cavity, each of the at least one fluid-providing channel pointing, in a fluid-providing direction, toward an interior of the flushing cavity, each fluid-providing direction pointing away from a centroid of the flushing cavity.

In some embodiments, each fluid-providing direction may include a directional component oriented to cause fluid rotation about the centroid of the flushing cavity. In some embodiments, each fluid-providing direction may obliquely enter the flushing cavity.

In some embodiments, the at least one fluid-providing channel may include a plurality of fluid-providing channels. In some embodiments, the fluid-providing channels of the plurality of fluid-providing channels may be circumferentially arranged about the centroid of the flushing cavity. In some embodiments, the fluid-providing direction of each of the plurality of fluid-providing channels collectively are a plurality of fluid-providing directions, and the plurality of fluid-providing directions may be oriented to cause fluid rotation about the centroid of the flushing cavity. In some embodiments, the plurality of fluid-providing channels may be aligned on a two-dimensional cross-section of the flushing cavity. In some embodiments, a centroid of the two-dimensional cross-section of the flushing cavity and the centroid of the flushing cavity may appear at a same location at least as viewed along a line passing through the centroid of the two-dimensional cross-section, the line perpendicular to a plane in which the two-dimensional cross-section resides.

In some embodiments, at least a portion of the flushing cavity may be defined by at least part of a body of revolution having an axis of revolution. In some embodiments, the at least one fluid-providing channel may include a plurality of fluid-providing channels, the plurality of fluid-providing channels circumferentially arranged about the axis of revolution. In some embodiments, the axis of revolution may extend through the centroid of the flushing cavity. In some embodiments, wherein the fluid-providing directions of the plurality of fluid-providing channels may point in a same rotational direction about the axis of revolution.

In some embodiments, the flushing cavity may be provided at least in part by a first interior chamber of the enclosure, and the enclosure may include a second interior chamber. In some embodiments, the plurality of fluid-providing channels may be located between the first interior chamber and the second interior chamber. In some embodiments, the second interior chamber may circumferentially surround at least part of the first interior chamber.

In some embodiments, the at least one fluid-providing channel may include a plurality of fluid-providing channels, and the plurality of fluid-providing channels may be aligned on a two-dimensional cross-section of the flushing cavity. In some embodiments, each fluid-providing channel of the plurality of fluid-providing channels forms a fluid-providing port leading into the flushing cavity. A first line may extend through a centroid of the fluid-providing port along the fluid-providing direction of the fluid-providing channel, and for each fluid-providing channel of the plurality of fluid-providing channels, an angle between the first line and a second line extending through the centroid of the fluid-providing port of the fluid-providing channel and the centroid of the flushing cavity may between 10 and 30 degrees. In some embodiments, the angle may be the same for at least two fluid-providing channels of the plurality of fluid-providing channels. In some embodiments, a centroid of the two-dimensional cross-section of the flushing cavity and the centroid of the flushing cavity may appear at a same location at least as viewed along a line passing through the centroid of the two-dimensional cross-section, the line perpendicular to a plane in which the two-dimensional cross-section resides. In some embodiments, the two-dimensional cross-section may reside on a cross-sectional plane, and, for each fluid-providing channel of the plurality of fluid-providing channels, the second line may extend along the cross-sectional plane through the centroid of the fluid-providing port of the fluid-providing channel and the centroid of the flushing cavity.

In some embodiments, the at least one fluid-providing channel may include a plurality of fluid-providing channels, and the plurality of fluid-providing channels may be aligned on a two-dimensional cross-section of the flushing cavity. In some embodiments, each fluid-providing channel of the plurality of fluid-providing channels forms a fluid-providing port leading into the flushing cavity. A first line may extend through a centroid of the fluid-providing port along the fluid-providing direction of the fluid-providing channel, and, for each fluid-providing channel of the plurality of fluid-providing channels, an angle between the first line and a second line extending through the centroid of the fluid-providing port of the fluid-providing channel and the centroid of the flushing cavity may be between 10 and 60 degrees. In some embodiments, the angle may be the same for at least two fluid-providing channels of the plurality of fluid-providing channels. In some embodiments, a centroid of the two-dimensional cross-section of the flushing cavity and the centroid of the flushing cavity may appear at a same location at least as viewed along a line passing through the centroid of the two-dimensional cross-section, the line perpendicular to a plane in which the two-dimensional cross-section resides. In some embodiments, the two-dimensional cross-section may reside on a cross-sectional plane, and, for each fluid-providing channel of the plurality of fluid-providing channels, the second line may extend along the cross-sectional plane through the centroid of the fluid-providing port of the fluid-providing channel and the centroid of the flushing cavity.

In some embodiments, the at least one fluid-providing channel may include a plurality of fluid-providing channels. In some embodiments, the plurality of fluid-providing channels are circumferentially arranged about an axis extending through the centroid of the flushing cavity, and the fluid-providing directions of the plurality of fluid-providing channels point in a same rotational direction about the axis.

In some embodiments, the at least one fluid-providing channel may be oriented to generate a fluid vortex within the flushing cavity at least in a state in which fluid is provided to the flushing cavity. In some embodiments, the flushing cavity may be configured to cause the fluid vortex to spin around the centroid of the flushing cavity when the fluid vortex is generated within the flushing cavity at least in the state in which fluid is provided to the flushing cavity. In some embodiments, the at least one fluid-providing channel may include a plurality of fluid-providing channels. The plurality of fluid-providing channels may be aligned on a two-dimensional cross-section of the flushing cavity, and the flushing cavity may be configured to cause the fluid vortex to spin around a centroid of the two-dimensional cross-section of the flushing cavity.

In some embodiments, the at least one fluid-providing channel may include a plurality of fluid-providing channels. In some embodiments, the plurality of fluid-providing channels may be collectively oriented to generate a fluid vortex within the flushing cavity at least in a state in which fluid is provided to the flushing cavity. In some embodiments, the generated fluid vortex helically spins about an axis at least in the state in which fluid is provided to the flushing cavity, and the fluid-providing directions of the plurality of fluid-providing channels may point in a same rotational direction about the axis. In some embodiments, the enclosure may include a fluid supply input port. The enclosure fluidically couples the fluid supply input port to all of the plurality of fluid-providing channels of the enclosure, and the fluid supply input port may be spaced from the axis, according to some embodiments.

In some embodiments, the at least one fluid-providing channel may include a plurality of fluid-providing channels. In some embodiments, the enclosure may include a fluid supply input port. The enclosure may fluidically couple the fluid supply input port to the plurality of fluid-providing channels of the enclosure. In some embodiments, the flushing cavity is provided at least in part by a first interior chamber of the enclosure, and the enclosure includes a second interior chamber. The second interior chamber may be located at least in part between the first interior chamber and the fluid supply input port, according to some embodiments. In some embodiments, the fluid supply input port is provided by a fluid input channel, and a longitudinal axis of the fluid input channel may extend toward the flushing cavity along a particular direction that does not intersect the centroid of the flushing cavity. In some embodiments, a fluid input channel providing the fluid supply input port may be arranged to cause fluid to enter an interior volume within the enclosure along a particular direction that does not intersect the centroid of the flushing cavity. In some embodiments, each of at least some of the fluid-providing directions may be different than a particular direction in which a fluid input channel providing the fluid supply input port is configured to cause fluid to enter the interior volume within the enclosure. In some embodiments, a fluid input channel providing the fluid supply input port is arranged to cause fluid to enter an interior volume within the enclosure along a particular direction, and the particular direction and the respective fluid-providing directions may be oriented in a same rotational direction. In some embodiments, the plurality of fluid-providing channels are circumferentially arranged about an axis, and a fluid input channel providing the fluid supply input port may be arranged to cause fluid to enter an interior volume within the enclosure along a particular direction that has a directional component that is transversely oriented to a direction in which the axis extends. In some embodiments, the axis may extend through the centroid of the flushing cavity. In some embodiments, at least a portion of the flushing cavity is defined by at least part of a body of revolution having an axis of revolution, and a fluid input channel providing the fluid supply input port may be arranged to cause fluid to enter an interior volume within the enclosure along a particular direction that has a directional component that is transversely oriented to a direction in which the axis of revolution extends. In some embodiments, the axis of revolution may extend through the centroid of the flushing cavity. In some embodiments, the plurality of fluid-providing channels are collectively oriented to generate a fluid vortex within the flushing cavity at least in a state in which fluid is provided to the flushing cavity, the generated fluid vortex helically spinning about an axis, and a channel fluidically coupled to the fluid supply input port may be arranged to cause fluid to enter an interior volume within the enclosure along a particular direction that has a directional component that is transversely oriented to a direction in which the axis extends. In some embodiments, the enclosure may include a fluid output port fluidically coupled to the flushing cavity to allow removal from the flushing cavity of at least some of the fluid provided to the flushing cavity. In some embodiments, a fluid input channel providing the fluid supply input port and a fluid output channel providing the fluid output port may be arranged such that a particular direction in which fluid enters an interior volume within the enclosure via the fluid input channel providing the fluid supply input port and a particular direction in which fluid is removed from the flushing cavity via the fluid output channel providing the fluid output port are non-parallel directions. In some embodiments, the plurality of fluid-providing channels may collectively be oriented to generate a fluid vortex within the flushing cavity at least in a state in which fluid is provided to the flushing cavity, the generated fluid vortex helically spinning about an axis, and one of (a) the particular direction in which fluid enters the interior volume within the enclosure via the fluid input channel providing the fluid supply input port and (b) the particular direction in which fluid is removed from the flushing cavity via the fluid output channel providing the fluid output port is not parallel to a direction in which the axis extends while the other of (a) and (b) is parallel to the direction in which the axis extends.

In some embodiments, the enclosure may include a fluid output port fluidically coupled to the flushing cavity to allow removal from the flushing cavity of at least some of the fluid provided to the flushing cavity, and at least a first fluid-providing channel of the at least one fluid-providing channel and a fluid output channel providing the fluid output port may be arranged such that a particular direction in which fluid enters the flushing cavity within the enclosure via the first fluid-providing channel and a particular direction in which fluid is removed from the flushing cavity via the fluid output channel providing the fluid output port are non-parallel directions.

In some embodiments, the enclosure may include a medical device structure-receiving port arranged to receive the structure of the medical device to allow entry of at least a portion thereof into the flushing cavity. In some embodiments, the medical device structure-receiving port may be provided by a medical device structure-receiving channel that is positioned to constrain entry of the portion of the structure into the flushing cavity along a direction that does not point toward the centroid of the flushing cavity. In some embodiments, the structure of the medical device is selectively moveable between a delivery configuration in which at least the structure of the medical device is sized to be percutaneously deliverable to a bodily cavity and a deployed configuration in which at least the structure of the medical device is sized to be too large to be percutaneously deliverable to the bodily cavity, and the medical device structure-receiving port may be sized to receive the structure of the medical device in a state in which the structure of the medical device is in the delivery configuration, but not the deployed configuration. In some embodiments, the structure of the medical device is selectively moveable between a delivery configuration and a deployed configuration, at least a portion of the structure of the medical device having a dimension that is smaller in the delivery configuration than a corresponding dimension of the at least the portion of the structure of the medical device in the deployed configuration, and the medical device structure-receiving port may be sized to receive the structure of the medical device in a state in which the structure of the medical device is in the delivery configuration, but not the deployed configuration.

In some embodiments, the enclosure may include a first housing and a second housing physically coupled to the first housing, and at least a portion of the plurality of fluid-providing channels may be provided in the first housing, the second housing, or each of both the first housing and the second housing. In some embodiments, the enclosure may include a first housing and a second housing physically coupled to the first housing. At least a portion of the plurality of fluid-providing channels is provided in the first housing, the second housing, or each of both the first housing and the second housing, and the fluid supply input port, the fluid output port, or each of the fluid supply input port and the fluid output port may be provided, at least in part, by the first housing or the second housing, but not both the first housing and the second housing.

In some embodiments, the enclosure may include a first housing and a second housing physically coupled to the first housing, and each of at least a first fluid-providing channel of the at least one fluid-providing channel may be defined at least in part by each of the first housing and the second housing.

Various apparatuses may be defined by combinations (which includes subcombinations) of the apparatuses described above.

In some embodiments, a medical device flushing apparatus may be summarized as including an enclosure including an interior surface defining at least part of a flushing cavity, the interior surface including a structure-receiving port configured to receive a distal end structure of a medical device, at least part of the medical device configured to be percutaneously delivered toward a bodily cavity distal end structure first, and at least one fluid-providing channel arranged to obliquely interrupt the interior surface of the enclosure, the at least one fluid-providing channel configured to provide fluid to the flushing cavity.

In some embodiments, the at least one fluid-providing channel may include a plurality of fluid-providing channels. In some embodiments, fluid-providing channels of the plurality of fluid-providing channels may be circumferentially arranged about a centroid of the flushing cavity. In some embodiments, the plurality of fluid-providing channels may be aligned on a two-dimensional cross-section of the flushing cavity.

In some embodiments, at least a portion of the flushing cavity may be defined by at least part of a body of revolution having an axis of revolution. In some embodiments, the at least one fluid-providing channel may include a plurality of fluid-providing channels, the plurality of fluid-providing channels circumferentially arranged about the axis of revolution. In some embodiments, each of the plurality of fluid-providing channels may point, in a fluid-providing direction, toward an interior of the flushing cavity, the fluid-providing directions of the plurality of fluid-providing channels pointing in a same rotational direction about the axis of revolution.

In some embodiments, the at least one fluid-providing channel may include a plurality of fluid-providing channels. In some embodiments, the flushing cavity is provided at least in part by a first interior chamber of the enclosure, and the enclosure includes a second interior chamber, and the plurality of fluid-providing channels may be located at least in part between the first interior chamber and the second interior chamber. In some embodiments, the second interior chamber may circumferentially surround at least part of the first interior chamber. In some embodiments, the structure-receiving port may be arranged to not interrupt an internal surface of the second interior chamber. In some embodiments, a structure-receiving channel that provides the structure-receiving port does not extend through the second interior chamber. In some embodiments, each fluid-providing channel of the plurality of fluid-providing channels may point, in a fluid-providing direction, toward an interior of the flushing cavity. The plurality of fluid-providing channels may be circumferentially arranged about an axis extending through a centroid of the flushing cavity, and the fluid-providing directions of the plurality of fluid-providing channels may point in a same rotational direction about the axis.

In some embodiments, the at least one fluid-providing channel may be oriented to generate a fluid vortex within the flushing cavity at least in a state in which fluid is provided to the flushing cavity.

In some embodiments, the at least one fluid-providing channel may include a plurality of fluid-providing channels. In some embodiments, the plurality of fluid-providing channels may collectively be oriented to generate a fluid vortex within the flushing cavity at least in a state in which fluid is provided to the flushing cavity. In some embodiments, each of the plurality of fluid-providing channels may point, in a fluid-providing direction, toward an interior of the flushing cavity, and the generated fluid vortex helically spins about an axis centrally located in the generated fluid vortex at least in the state in which fluid is provided to the flushing cavity. The fluid-providing directions of the plurality of fluid-providing channels may point in a same rotational direction about the axis. In some embodiments, the enclosure may include a fluid supply input port, and the enclosure fluidically couples the fluid supply input port to the plurality of fluid-providing channels of the enclosure. The fluid supply input port may be spaced from the axis. In some embodiments, the structure-receiving port may be spaced from the axis.

In some embodiments, the at least one fluid-providing channel may include a plurality of fluid-providing channels. In some embodiments, the enclosure may include a fluid supply input port. The enclosure may fluidically couple the fluid supply input port to all of the plurality of fluid-providing channels of the enclosure, and a fluid input channel providing the fluid supply input port may be arranged to cause fluid to enter an interior volume within the enclosure along a particular direction that does not intersect a centroid of the flushing cavity.

In some embodiments, the at least one fluid-providing channel may include a plurality of fluid-providing channels. In some embodiments, the enclosure may include a fluid supply input port. The enclosure may fluidically couple the fluid supply input port to the plurality of fluid-providing channels of the enclosure, and each fluid-providing channel of the plurality of fluid-providing channels may point, in a fluid-providing direction, toward an interior of the flushing cavity. Each of at least some of the fluid-providing directions may be different than a particular direction in which a fluid input channel providing the fluid supply input port is configured to cause fluid to enter an interior volume within the enclosure. In some embodiments, the fluid-providing directions and the particular direction in which the fluid input channel providing the fluid supply input port is configured to cause fluid to enter the interior volume within the enclosure may be oriented in a same rotational direction. In some embodiments, at least a portion of the flushing cavity may be defined by at least part of a body of revolution having an axis of revolution, and the particular direction in which the fluid input channel providing the fluid supply input port is configured to cause fluid to enter the interior volume within the enclosure may have a directional component that is transversely oriented to a direction in which the axis of revolution extends. In some embodiments, the plurality of fluid-providing channels may be collectively oriented to generate a fluid vortex within the flushing cavity at least in a state in which fluid is provided to the flushing cavity, the generated fluid vortex helically spinning about an axis, and the particular direction in which the fluid input channel providing the fluid supply input port is configured to cause fluid to enter the interior volume within the enclosure may have a directional component that is transversely oriented to a direction in which the axis extends. In some embodiments, the enclosure may include a fluid output port fluidically coupled to the flushing cavity to allow removal from the flushing cavity of at least some of the fluid provided to the flushing cavity. In some embodiments, the fluid input channel providing the fluid supply input port and a fluid output channel providing the fluid output port may be arranged such that the particular direction in which fluid enters the interior volume within the enclosure via the fluid input channel providing the fluid supply input port and a particular direction in which fluid is removed from the flushing cavity via the fluid output channel providing the fluid output port are non-parallel directions. In some embodiments, the plurality of fluid-providing channels may be collectively oriented to generate a fluid vortex within the flushing cavity at least in a state in which fluid is provided to the flushing cavity, the generated fluid vortex helically spinning about an axis. In some embodiments, one of (a) the particular direction in which fluid enters the interior volume within the enclosure via the fluid input channel providing the fluid supply input port and (b) the particular direction in which fluid is removed from the flushing cavity via the fluid output channel providing the fluid output port is not parallel to a direction in which the axis extends while the other of (a) and (b) is parallel to the direction in which the axis extends.

In some embodiments, the enclosure may include a fluid output port fluidically coupled to the flushing cavity to allow removal from the flushing cavity of at least some of the fluid provided to the flushing cavity. At least a first fluid-providing channel of the at least one fluid-providing channel and a fluid output channel providing the fluid output port may be arranged such that a particular direction in which fluid enters the flushing cavity within the enclosure via the first fluid-providing channel and a particular direction in which fluid is removed from the flushing cavity via the fluid output channel providing the fluid output port are non-parallel directions.

In some embodiments, a structure-receiving channel providing the structure-receiving port and the at least one fluid-providing channel may be arranged such that a particular direction in which the distal end structure of the medical device enters the flushing cavity via the structure-receiving channel providing the structure-receiving port and a particular direction in which fluid enters the flushing cavity via a first fluid-providing channel of the at least one fluid-providing channel are non-parallel directions.

In some embodiments, the structure-receiving port may be provided by a structure-receiving channel that is positioned to constrain entry of the distal end structure of the medical device into the flushing cavity along a direction that does not point toward a centroid of the flushing cavity.

In some embodiments, the distal end structure of the medical device is selectively moveable between a delivery configuration in which at least the distal end structure of the medical device is sized to be percutaneously deliverable to a bodily cavity and a deployed configuration in which at least the distal end structure of the medical device is sized to be too large to be percutaneously deliverable to the bodily cavity. The structure-receiving port may be sized to receive the distal end structure of the medical device in a state in which the distal end structure of the medical device is in the delivery configuration, but not the deployed configuration.

In some embodiments, the distal end structure of the medical device may be selectively moveable between a delivery configuration and a deployed configuration, at least a portion of the distal end structure of the medical device having a dimension that is smaller in the delivery configuration than a corresponding dimension of the at least the portion of the distal end structure of the medical device in the deployed configuration.

Various apparatuses may be defined by combinations (which includes subcombinations) of the apparatuses described above.

Various embodiments of the present invention may include systems, devices, or machines that are or include combinations or subsets of any one or more of the systems, devices, or machines and associated features thereof described herein.

Further, all or part of any one or more of the systems, devices, or machines discussed herein or combinations or sub-combinations thereof may implement or execute all or part of any one or more of the processes or methods discussed herein or combinations or sub-combinations thereof.

For example, in some embodiments, a medical device flushing method may be summarized as providing a flushing chamber, a portion of the medical device received within an internal cavity of the flushing chamber and generating a fluid vortex within the internal cavity of the flushing chamber by causing fluid movement within the internal cavity of the flushing chamber at least independently of any movement of the portion of the medical device while the portion of the medical device is received within the internal cavity of the flushing chamber, the fluid vortex causing circumferential fluid flow that encircles the portion of the medical device received within the internal cavity of the flushing chamber. The medical device flushing method may include removing the portion of the medical device from the internal cavity of the flushing chamber at least after initiation of the generating the fluid vortex within the internal cavity of the flushing chamber.

In some embodiments, the portion of the medical device may be a distal end structure of a medical device, the medical device configured to be percutaneously delivered toward a bodily cavity distal end structure first.

In some embodiments, the portion of the medical device may include a structure comprising a plurality of transducers. In some embodiments, at least some of the transducers may be operable to selectively emit tissue ablative energy.

In some embodiments, the portion of the medical device may include a structure that is selectively moveable between a delivery configuration in which the structure is sized to be percutaneously deliverable toward a bodily cavity located within a patient, and a deployed configuration in which the structure is sized too large to be percutaneously deliverable toward the bodily cavity. In some embodiments, the structure may include a plurality of elongate members, a set of one or more transducers located on each of at least some of the plurality of elongate members. In some embodiments, the structure may include an expandable balloon.

In some embodiments, the portion of the medical device may include a structure that is selectively moveable between a delivery configuration and a deployed configuration, at least a portion of the structure of the medical device having a dimension that is smaller in the delivery configuration than a corresponding dimension of the at least the portion of the structure of the medical device in the deployed configuration. In some embodiments, the generating the fluid vortex within the internal cavity of the flushing chamber occurs at least independently of any movement of the portion of the medical device while the structure is received within the internal cavity of the flushing chamber in the deployed configuration. In some embodiments, the portion of the medical device received within the internal cavity of the flushing chamber may include the structure positioned in the deployed configuration. In some embodiments, the medical device flushing method may include moving the structure between the delivery configuration and the deployed configuration within the internal cavity of the flushing chamber. In some embodiments, the medical device flushing method may include moving the structure between the delivery configuration and the deployed configuration within the internal cavity of the flushing chamber at least after the initiation of the generating the fluid vortex within the internal cavity of the flushing chamber. In some embodiments, the medical device flushing method may include moving the structure between the delivery configuration and the deployed configuration within the internal cavity of the flushing chamber at least before the removing the portion of the medical device from the internal cavity of the flushing chamber.

In some embodiments, the portion of the medical device may include an implant.

In some embodiments, the medical device flushing method may include injecting fluid into the internal cavity of the flushing chamber to cause, at least in part, the generating the fluid vortex within the internal cavity of the flushing chamber. In some embodiments, the injecting fluid into the internal cavity of the flushing chamber may include injecting fluid into the internal cavity of the flushing chamber via at least one fluid-providing channel interrupting an interior surface of the flushing chamber, each of the at least one fluid-providing channel pointing, in a fluid-providing direction, toward the interior of the internal cavity of the flushing chamber, each fluid-providing direction pointing away from a centroid of the internal cavity of the flushing chamber. In some embodiments, the at least one fluid-providing channel may include a plurality of fluid-providing channels. In some embodiments, fluid-providing channels of the plurality of fluid-providing channels may be circumferentially arranged about the centroid of the internal cavity of the flushing chamber.

In some embodiments, the medical device flushing method may include removing fluid from the internal cavity of the flushing chamber during the generating the fluid vortex within the internal cavity of the flushing chamber. In some embodiments, the medical device flushing method may include removing fluid from the internal cavity of the flushing chamber via a fluid output port in fluid communication with the internal cavity of the flushing chamber, the fluid output port positioned to be encircled by the circumferential fluid flow.

In some embodiments, the medical device flushing method may include, during the generating the fluid vortex within the internal cavity of the flushing chamber, concurrently supplying fluid to the internal cavity of the flushing chamber and removing fluid from the internal cavity of the flush chamber.

In some embodiments, the fluid vortex rotates about an axis, and the medical device flushing method may include supplying fluid to a first region of the internal cavity of the flushing chamber and removing fluid from a second region of the internal cavity of the flushing chamber, the first region of the internal cavity of the flushing chamber located further radially outward from the axis than the second region of the internal cavity of the flushing chamber. In some embodiments, the supplying fluid to the first region of the internal cavity of the flushing chamber and the removing fluid from the second region of the internal cavity of the flushing chamber occur concurrently.

In some embodiments, the medical device flushing method may include rotating a moveable member to cause the generating the fluid vortex within the internal cavity of the flushing chamber, the moveable member not forming any portion of the medical device. In some embodiments, the moveable member may located within the internal cavity of the flushing chamber at least during the generating the fluid vortex within the internal cavity of the flushing chamber. In some embodiments, the moveable member may include an impeller.

In some embodiments, the medical device flushing method may include operating a motor operatively coupled to the flushing chamber to cause, at least in part, the generating the fluid vortex within the internal cavity of the flushing chamber, the motor driven electrically, pneumatically or hydraulically.

In some embodiments, the medical device flushing method may include delivering energy to a mechanical apparatus operatively coupled to the flushing chamber to facilitate the generating the fluid vortex within the internal cavity of the flushing chamber, the mechanical apparatus not forming any portion of the medical device.

In some embodiments, the causing fluid movement within the internal cavity of the flushing chamber may include providing fluid into the internal cavity of the flushing chamber via a plurality of fluid-providing channels. In some embodiments, each fluid-providing channel of the plurality of fluid-providing channels point, in a fluid-providing direction, toward the internal cavity of the flushing chamber, and the generated fluid vortex helically spins about an axis. The fluid-providing directions of the plurality of fluid-providing channels may point in a same rotational direction about the axis, according to some embodiments. In some embodiments, the portion of the medical device is received within the internal cavity of the flushing chamber via a structure-receiving port. The structure-receiving port may be spaced from the axis, according to some embodiments. In some embodiments, the causing fluid movement within the internal cavity of the flushing chamber includes providing fluid into the internal cavity via a fluid supply input port, and an enclosure fluidically couples the fluid supply input port to all of the plurality of fluid-providing channels. A fluid input channel providing the fluid supply input port may be arranged to cause fluid to enter an interior volume within the enclosure along a particular direction that does not intersect a centroid of the internal cavity of the flushing chamber, according to some embodiments. In some embodiments, the causing fluid movement within the internal cavity of the flushing chamber includes providing fluid into the internal cavity via at least a fluid input channel providing a fluid supply input port, and via at least an enclosure that fluidically couples the fluid supply input port to the plurality of fluid-providing channels. Each of the plurality of fluid-providing channels points, in a fluid-providing direction, toward an interior of the internal cavity of the flushing chamber. In some embodiments, each of at least some of the fluid-providing directions may be different than a particular direction in which the fluid input channel providing the fluid supply input port is configured to cause fluid to enter an interior volume within the enclosure. In some embodiments, at least a portion of the internal cavity of the flushing chamber may be defined by at least part of a body of revolution having an axis of revolution. In some embodiments, the particular direction in which the fluid input channel providing the fluid supply input port is configured to cause fluid to enter the interior volume within the enclosure has a directional component that may be transversely oriented to a direction in which the axis of revolution extends. In some embodiments, the generated fluid vortex helically spins about an axis, and the particular direction in which the fluid input channel providing the fluid supply input port is configured to cause fluid to enter the interior volume within the enclosure may have a directional component that is transversely oriented to a direction in which the axis extends. In some embodiments, the medical device flushing method may include removing from the internal cavity of the flushing chamber at least some fluid provided into the internal cavity of the flushing chamber via a fluid output port fluidically coupled to the internal cavity of the flushing chamber. In some embodiments, the fluid input channel providing the fluid supply input port and a fluid output channel providing the fluid output port may be arranged such that a particular direction in which fluid enters the interior volume within the enclosure via the fluid input channel providing the fluid supply input port and a particular direction in which fluid is removed from the internal cavity of the flushing chamber via the fluid output channel providing the fluid output port are non-parallel directions. In some embodiments, the plurality of fluid-providing channels are collectively oriented to generate the fluid vortex within the internal cavity of the flushing chamber, the generated fluid vortex helically spinning about an axis, and one of (a) the particular direction in which fluid enters the interior volume within the enclosure via the fluid input channel providing the fluid supply input port and (b) the particular direction in which fluid is removed from the internal cavity of the flushing chamber via the fluid output channel providing the fluid output port is not parallel to a direction in which the axis extends while the other of (a) and (b) is parallel to the direction in which the axis extends.

In some embodiments, the medical device flushing method may include removing from the internal cavity of the flushing chamber at least some fluid provided into the internal cavity of the flushing chamber via a fluid output port fluidically coupled to the internal cavity of the flushing chamber. In some embodiments, the causing fluid movement within the internal cavity of the flushing chamber may include providing fluid into the internal cavity of the flushing chamber via at least one fluid-providing channel, and at least a first fluid-providing channel of the at least one fluid-providing channel and a fluid output channel providing the fluid output port may be arranged such that a particular direction in which fluid enters the internal cavity of the flushing chamber and a particular direction in which fluid is removed from the internal cavity of the flushing chamber via the fluid output channel providing the fluid output port are non-parallel directions.

In some embodiments, the portion of the medical device is received within the internal cavity of the flushing chamber via a structure-receiving channel providing a structure-receiving port. In some embodiments, the causing fluid movement within the internal cavity of the flushing chamber may include providing fluid into the internal cavity of the flushing chamber via at least one fluid-providing channel, and the structure-receiving channel providing the structure-receiving port and the at least one fluid-providing channel may be arranged such that a particular direction in which the portion of the medical device enters the internal cavity of the flushing chamber via the structure-receiving channel providing the structure-receiving port and a particular direction in which fluid enters the internal cavity of the flushing chamber via a first fluid-providing channel of the at least one fluid-providing channel are non-parallel directions.

In some embodiments, the portion of the medical device is received within the internal cavity of the flushing chamber via a structure-receiving channel providing a structure-receiving port, and the structure-receiving channel may positioned to constrain entry of the portion of the medical device into the internal cavity of the flushing chamber along a direction that does not point toward a centroid of the internal cavity of the flushing chamber.

Further, any of all or part of one or more of the methods or processes and associated features thereof discussed herein may be implemented or executed by all or part of a device system, apparatus, or machine, such as all or a part of any of one or more of the systems, apparatuses, or machines described herein or a combination or sub-combination thereof.

### BRIEF DESCRIPTION OF THE DRAWINGS

It is to be understood that the attached drawings are for purposes of illustrating aspects of various embodiments and may include elements that are not to scale.

FIG. 1 is a cutaway diagram of a heart showing an example of a medical device therein in a deployed or expanded configuration, the medical device suitable for undergoing flushing of undesired fluid, e.g., air, by various embodiments of one or more of the inventive flushing apparatuses described herein prior to insertion into the heart, according to some embodiments of the present invention.

FIG. 2 is a partial schematic view of an example of a medical device, which may represent the medical device shown in FIG. 1, but in a delivery of unexpanded configuration, according to some embodiments.

FIG. 3 is a partial schematic view of an example of a medical device, which may represent the medical device shown in FIG. 2, but in a deployed or expanded configuration, according to some embodiments.

FIG. 4A illustrates a flushing chamber enclosure of a medical device flushing apparatus, according to some embodiments.

FIG. 4B illustrates the flushing chamber enclosure of at least FIG. 4A, but in separated form to reveal internal structure of the flushing chamber enclosure, according to some embodiments.

FIG. 4C is a cutaway diagram of the flushing chamber enclosure of at least FIG. 4A, according to some embodiments.

FIG. 4D is the same cutaway diagram of FIG. 4C, but with reference numeral and other descriptive differences in order to illustrate different aspects of the flushing chamber enclosure, according to some embodiments.

FIG. 4E illustrates a medical device flushing apparatus in disconnected and connected forms with respect to the flushing chamber enclosure of at least FIG. 4A and a loading assembly, according to some embodiments.

FIGS. 4F, 4G, 4H are cutaway diagrams of at least the medical device flushing apparatus of FIG. 4E, illustrating various states of insertion and expansion of a medical device within the medical device flushing apparatus, according to some embodiments.

FIG. 4I is a cutaway diagram of a medical device flushing apparatus, according to some embodiments.

FIGS. 4J and 4K are cutaway diagrams of at least a medical device flushing apparatus of FIG. 4E with different types of medical devices therein, according to some embodiments.

FIG. 4L is a cutaway diagram of a medical device flushing apparatus, according to some embodiments.

FIG. 4M is a cutaway diagram of a medical device flushing apparatus, according to some embodiments.

FIG. 5 illustrates a medical device flushing method, according to some embodiments.

### DETAILED DESCRIPTION

Various embodiments of the present invention address the above-discussed need and provide technical solutions in the art with inventive medical device flushing systems and methods. In some embodiments, such systems include an improved structure of an enclosure of a flushing cavity, and such methods include inserting a manipulable portion of an elongate shaft member of a catheter device system into the flushing cavity. In some embodiments, while the manipulable portion of the elongate shaft member is inserted in the flushing cavity, flushing fluid is provided to and, in various embodiments, removed from the enclosure. One or more fluid-providing channels of the enclosure are configured in an efficient and effective arrangement to consequently generate a vortex of flushing fluid within the flushing cavity to efficiently and effectively flush or otherwise remove undesired fluid (e.g., air) therefrom. In some embodiments, the manipulable portion is inserted into the flushing fluid within the flushing cavity while at least the elongate shaft member is in a substantially horizontal orientation, which can be more beneficial than conventional vertical flushing orientations at least because the horizontal flushing arrangement is easier to physically handle by a user. It should be noted that the invention is not limited to these or any other examples or improvements, which are referred to for purposes of illustration only.

In this regard, in the descriptions herein, certain specific details are set forth in order to provide a thorough understanding of various embodiments of the invention. However, one skilled in the art will understand that the invention may be practiced at a more general level without one or more of these details. In other instances, well-known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily all referring to one embodiment or a same embodiment. Furthermore, the particular features, structures or characteristics of different embodiments may be combined in any suitable manner to form one or more other embodiments.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects.

Further, the phrase “at least” is or may be used herein at times merely to emphasize the possibility that other elements may exist besides those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase ‘based at least on A’ includes A as well as the possibility of one or more other additional elements besides A. In the same manner, the phrase ‘based on A’ includes A, as well as the possibility of one or more other additional elements besides A. However, the phrase ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase ‘configured only to A’ means a configuration to perform only A.

The word “device”, the word “machine”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified, according to various embodiments that a device or machine or device system resides entirely within a same housing to exclude embodiments where the respective device, machine, or device system resides across different housings. The word “device” may equivalently be referred to as a “device system” in some embodiments.

Further, the phrase “in response to” may be used in this disclosure. For example, this phrase may be used in the following context, where an event A occurs in response to the occurrence of an event B. In this regard, such phrase includes, for example, that at least the occurrence of the event B causes or triggers the event A.

In some embodiments, the term “adjacent”, the term “proximate”, and the like refer at least to a sufficient closeness between the objects defined as adjacent, proximate, or the like, to allow the objects to interact in a designated way. For example, if object A performs an action on an adjacent or proximate object B, objects A and B would have at least a sufficient closeness to allow object A to perform the action on object B. In this regard, some actions may require contact between the associated objects, such that if object A performs such an action on an adjacent or proximate object B, objects A and B would be in contact, for example, in some instances or embodiments where object A needs to be in contact with object B to successfully perform the action. In some embodiments, the term “adjacent”, the term “proximate”, and the like additionally or alternatively refer to objects that do not have another substantially similar object between them. For example, object A and object B could be considered adjacent or proximate if they contact each other (and, thus, it could be considered that no other object is between them), or if they do not contact each other but no other object that is substantially similar to object A, object B, or both objects A and B, depending on the embodiment, is between them. In some embodiments, the term “adjacent”, the term “proximate”, and the like additionally or alternatively refer to at least a sufficient closeness between the objects defined as adjacent, proximate, and the like, the sufficient closeness being within a range that does not place any one or more of the objects into a different or dissimilar region, or does not change an intended function of any one or more of the objects or of an encompassing object that includes a set of the objects. Different embodiments of the present invention adopt different ones or combinations of the above definitions. Of course, however, the term “adjacent”, the term “proximate”, and the like are not limited to any of the above example definitions, according to some embodiments. In addition, the term “adjacent” and the term “proximate” do not have the same definition, according to some embodiments.

The term “proximal”, in the context of a proximal portion, proximal location, and the like of a medical device, includes, for example, the portion, location, and the like, being or being configured to be further away from a patient or portion of or region within a patient (e.g., a bodily cavity) intended to be treated or assessed by the medical device, as compared to a distal portion, location, and the like of the medical device, according to some embodiments. In some embodiments, the term “proximal”, in the context of a proximal portion, proximal location, and the like of a medical device, includes, for example, the portion, location, and the like, being or being configured to be delivered (e.g., percutaneously or intravascularly) toward a patient or portion of or region within a patient (e.g., a bodily cavity) intended to be treated or assessed by the medical device, after or behind a distal portion, location, and the like of the medical device. On the other hand, the term “distal”, in the context of a distal portion, distal location, and the like of a medical device, includes, for example, the portion, location, and the like, being or being configured to be closer to a patient or portion of or region within a patient (e.g., a bodily cavity) intended to be treated or assessed by the medical device, as compared to a proximal portion, location, and the like of the medical device, according to some embodiments. In some embodiments, the term “distal”, in the context of a distal portion, distal location, and the like of a medical device, includes, for example, the portion, location, and the like, being or being configured to be delivered (e.g., percutaneously or intravascularly) toward a patient or portion of or region within a patient (e.g., a bodily cavity) intended to be treated or assessed by the medical device, before or ahead of a proximal portion, location, and the like of the medical device.

The word “ablation” as used in this disclosure should be understood to include, for example, any disruption to certain properties of tissue. Most commonly, the disruption is to the electrical conductivity and is achieved by heating, which can be generated with resistive or radio-frequency (RF) techniques for example. However, any other technique for such disruption may be included when the term “ablation” is used, such as mechanical, chemical, electroporation or optical techniques.

The phrase “bodily opening” as used in this disclosure should be understood to include, for example, a naturally occurring bodily opening or channel or lumen; a bodily opening or channel or lumen or perforation formed by an instrument or tool using techniques that can include, but are not limited to, mechanical, thermal, electrical, chemical, and exposure or illumination techniques; a bodily opening or channel or lumen formed by trauma to a body; or various combinations of one or more of the above. Various elements having respective openings, lumens or channels and positioned within the bodily opening (e.g., a catheter sheath or catheter introducer) may be present in various embodiments. These elements may provide a passageway through a bodily opening for various devices employed in various embodiments.

The phrase “bodily cavity” as used in this disclosure should be understood to mean a cavity in a body. The bodily cavity may be a cavity provided in a bodily organ (e.g., an intra-cardiac cavity or chamber of a heart). The bodily cavity may be provided by a bodily vessel.

The word “tissue” as used in some embodiments in this disclosure should be understood to include, for example, any surface-forming tissue that is used to form a surface of a body or a surface within a bodily cavity, a surface of an anatomical feature or a surface of a feature associated with a bodily opening positioned in fluid communication with the bodily cavity. The tissue can include, for example, part or all of a tissue wall or membrane that defines a surface of the bodily cavity. In this regard, the tissue can form an interior surface of the cavity that surrounds a fluid within the cavity. In the case of cardiac applications, tissue can include, for example, tissue used to form an interior surface of an intra-cardiac cavity such as a left atrium or right atrium. In some embodiments, tissue is non-excised tissue. In some embodiments, the word tissue can refer to a tissue having fluidic properties (e.g., blood).

The term “transducer” as used in this disclosure should be interpreted broadly as any device capable of distinguishing between fluid and tissue, sensing temperature, creating heat, ablating tissue, measuring electrical activity of a tissue surface, stimulating tissue, or any combination thereof. A transducer can convert input energy of one form into output energy of another form. Without limitation, a transducer can include, for example, an electrode that functions as, or as part of, a sensing device included in the transducer, an energy delivery device included in the transducer, or both a sensing device and an energy delivery device included in the transducer. A transducer may be constructed from several parts, which may be discrete components or may be integrally formed.

The phrase “physically coupled” is intended to include, in some embodiments, a coupling between two objects that involves a coupling between the two objects where the two objects physical contact each other at least in one state of the coupling between the two objects. The phrases “fixedly coupled”, “permanently coupled”, and the like, are intended to include, in some embodiments, a secure coupling between two objects that, in some embodiments, does not involve or include a mechanism configured to release the coupling of the two objects. The phrases “removably coupled”, “detachably coupled”, and the like, are intended to include, in some embodiments, a coupling between two objects that, in some embodiments, allows such coupling to be repeatedly disengaged and re-engaged without damaging the coupling (if a distinct coupling mechanism exists, e.g., in contrast to an interference fit that relies on friction), without damaging either or both of the objects, or without damaging the coupling (if a distinct coupling mechanism exists). The phrase “operatively coupled” is intended to include, for example, a coupling between two objects that transmits force, energy, information, or other influence at least from one of the two objects to the other of the two objects. An operative coupling does not exclude the possibility of a physical or fixed coupling in addition to the operative coupling. Unless otherwise explicitly noted or required by context, for any connection or coupling, direct or indirect, between components, devices, or other physical objects described herein, different embodiments include different ones of the above-described coupling types for such components, devices, or other physical objects. For example, unless otherwise explicitly noted or required by context, if a first physical object is shown in the figures or described in this text as being connected or coupled, directly or indirectly, to a second physical object, some embodiments will have the first physical object fixedly coupled to the second physical object; other embodiments will have the first physical object permanently coupled to the second physical object; other embodiments will have the first physical object removably or detachably coupled to the second physical object; other embodiments will have the first physical object not fixedly or permanently coupled to the second physical object while having the first physical object physically coupled to the second physical object; other embodiments will have the first physical object not physically coupled or fixedly coupled to the second physical object, but will have the first physical object operatively coupled to the second physical object; etc.

The word “fluid”, as used in this disclosure, should be understood to include, for example, liquid or gas. In this regard, various embodiments of the present invention are described herein in the context of providing a flushing fluid to flush a medical device of undesired fluid (e.g., air). While it is quite common for the flushing fluid to be a liquid, such as saline, which is used to flush, e.g., undesired air from a medical device prior to insertion of the medical device into the body of a patient, the present inventors contemplate for the present invention that there may be certain types of desirable gas that may be used to flush undesirable fluid, such as air, from a medical device. For example, the present inventors contemplate for the present invention that carbon dioxide might be an option as a desirable flushing gas to flush undesired fluid (e.g., air) from a medical device. Accordingly, the present specification retains the usage of the phrase “flushing fluid” and the like with the thought that gas might be able to be used as a flushing fluid, even though many common implementations likely will utilize a flushing liquid, such as saline or heparinized saline.

In some embodiments, the phrases “fluid communication”, “fluidically communicate”, “fluidically coupled”, “fluidly communicate”, “fluidly coupled”, and the like, are intended to include, for example, a port or opening, of a physical object leading to a lumen or other internal cavity, where the port, opening, lumen, or internal cavity leads to a body (e.g., a source or drain) of a first fluid, such that (a) at least some of the first fluid moves or is able to move through (1) the port or opening into the lumen or other internal cavity, (2) the lumen or other internal cavity into the port or opening, or both (a)(1) and (a)(2); (b) at least some of a second fluid moves or is able to move through (1) the lumen or other internal cavity into the port or opening, (2) the port or opening into the lumen or other internal cavity, or both (b)(1) and (b)(2); or both (a) and (b). In some embodiments, the first fluid and the second fluid are the same. In some embodiments, the first fluid and the second fluid are different.

Various embodiments of catheter systems or catheter device systems are described herein. It should be noted that any catheter system described herein may also be referred to as a medical system or medical device system. Some of the described devices of such systems are medical devices that are percutaneously or intravascularly deployed. Some of the described devices are deployed through a bodily opening that is accessible without puncturing, cutting or otherwise perforating bodily tissue to create an access to the bodily opening. Some of the described devices employ transducer-based devices or device systems. Some of the described devices are moveable between a delivery or unexpanded configuration in which a portion of the device is sized, shaped, or both to be deliverable through a bodily opening leading to a bodily cavity, and an expanded or deployed configuration in which the portion of the device has a size, shape, or both too large to be deliverable through the bodily opening leading to the bodily cavity. An example of an expanded or deployed configuration is when the portion of the catheter system is in a state in which the portion of the catheter system is in its intended operational configuration. In a state in which the portion of the catheter system (e.g., at least the manipulable portion 200 or 300 discussed below) is inside a bodily cavity, the deployed configuration may be an expanded state of the portion of the catheter system in which the portion of the catheter system is configured to interact with tissue within the bodily cavity to perform a medical procedure on a patient. In a state in which the portion of the catheter system (e.g., at least the manipulable portion 200 or 300 discussed below) is not within any part of a patient's body (e.g., during testing of the catheter system, during flushing of the catheter system as discussed below, or some other pre-medical procedure or post-medical procedure state), the deployed configuration may be the expanded state of the portion of the catheter system in which the portion of the catheter system would normally be configured to interact with tissue if within a bodily cavity, but is not configured to interact with tissue since it is not within a bodily cavity. Another example of the expanded or deployed configuration is when the portion of the catheter system is being changed from the delivery configuration to the intended operational state to a point in which the portion of the device now has a size, shape, or both too large to be deliverable through the bodily opening leading to the bodily cavity. In some embodiments, at least a portion of at least one of the described devices has a dimension or size that is smaller in the delivery or unexpanded configuration than a corresponding dimension or size of the at least a portion of at least one of the described devices in the expanded or deployed configuration.

In some example embodiments, the catheter system includes transducers that sense characteristics (e.g., convective cooling, permittivity, force) that distinguish between fluid, such as a fluidic tissue (e.g., blood), and tissue forming an interior surface of the bodily cavity. Such sensed characteristics can allow a medical device system to map the cavity, for example using positions of openings or ports into and out of the cavity to determine a position or orientation (i.e., pose), or both of the portion of the device in the bodily cavity. In some example embodiments, the described devices are capable of ablating tissue in a desired pattern within the bodily cavity. In some example embodiments, the devices are capable of sensing characteristics (e.g., electrophysiological activity) indicative of whether an ablation has been successful. In some example embodiments, the devices are capable of providing stimulation (e.g., electrical stimulation) to tissue within the bodily cavity. Electrical stimulation may include pacing.

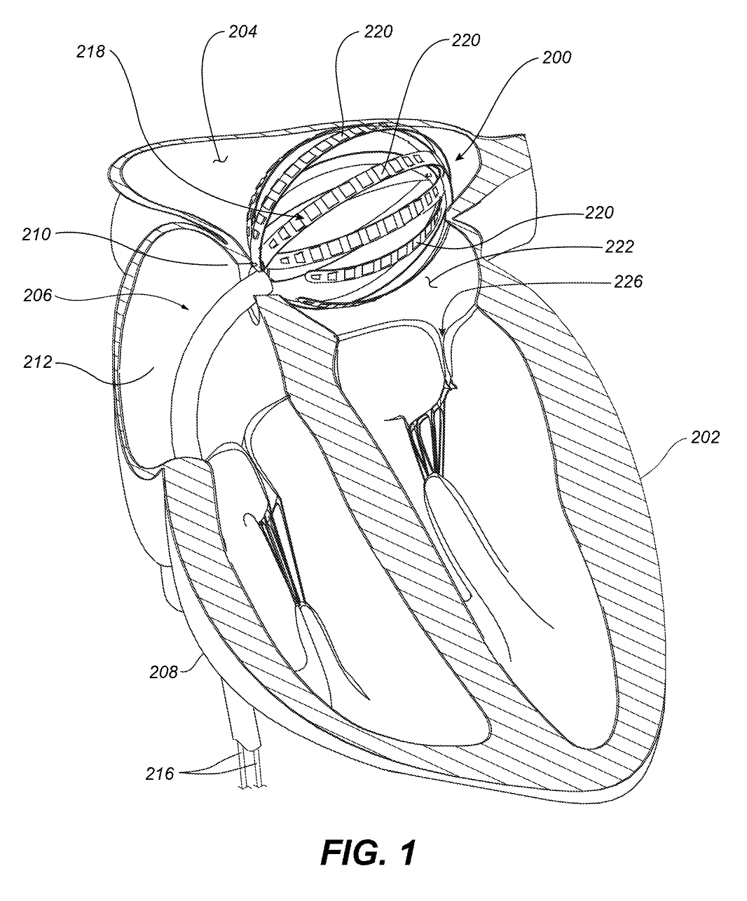

FIG. 1 shows a portion of a catheter system, according to some embodiments, such portion including a transducer-based device 200, which may be at least part of a medical device useful in investigating or treating a bodily organ, for example, a heart 202, according to some example embodiments. The transducer-based device 200 may also be referred to as a manipulable portion, due to its ability to have its size, shape, or both size and shape altered, according to some embodiments described below. Transducer-based device 200 can be percutaneously or intravascularly inserted into a portion of the heart 202, such as an intra-cardiac cavity like left atrium 204. The components of the transducer-based device 200 (as well as the transducer-based device 300, and the flushing apparatus 600, described below) may be sterile.

In the example of FIG. 1, the illustrated portion of the catheter system also includes a catheter 206, which may be inserted via the inferior vena cava 208 and may penetrate through a bodily opening in transatrial septum 210 from right atrium 212. In other embodiments, other paths may be taken.

Catheter 206 includes an elongate flexible rod or elongate shaft member appropriately sized to be delivered percutaneously or intravascularly. Various portions of catheter 206 may be steerable. Catheter 206 may include one or more lumens, for example within the elongate shaft member. The lumen(s) may carry one or more communications or power paths, or both. For example, the lumens(s) may carry one or more electrical conductors 216 (two shown in this embodiment). Electrical conductors 216 provide electrical connections to transducer-based device 200 that are accessible externally from a patient in which the transducer-based device 200 is inserted. The lumen(s) may carry various control elements (e.g., control lines) operatively coupling one or more actuators to a manipulable portion (e.g., manipulable portion 200).

In various embodiments, transducer-based device, or manipulable portion, 200 includes a frame or structure 218, which assumes an unexpanded configuration to facilitate delivery to left atrium 204. Structure 218 is expanded (i.e., shown in a deployed or expanded configuration in FIG. 1) upon delivery to left atrium 204 to position a plurality of transducers 220 (three called out in FIG. 1) proximate the interior surface formed by tissue 222 of left atrium 204. In this regard, it can be stated that one or more of the transducers 220 are moveable with one or more parts of the transducer-based device, or manipulable portion, 200. In some embodiments, at least some of the transducers 220 are used to sense a physical characteristic of a fluid (i.e., blood) or tissue 222, or both, that may be used to determine a position or orientation (i.e., pose), or both, of a portion of transducer-based device 200 within, or with respect to left atrium 204. For example, transducers 220 may be used to determine a location of pulmonary vein ostia (not shown) or a mitral valve 226, or both. In some embodiments, at least some of the transducers 220 may be used to selectively ablate portions of the tissue 222. For example, some of the transducers 220 may be used to ablate a pattern or path around various ones of the bodily openings, ports or pulmonary vein ostia, for instance to reduce or eliminate the occurrence of atrial fibrillation.

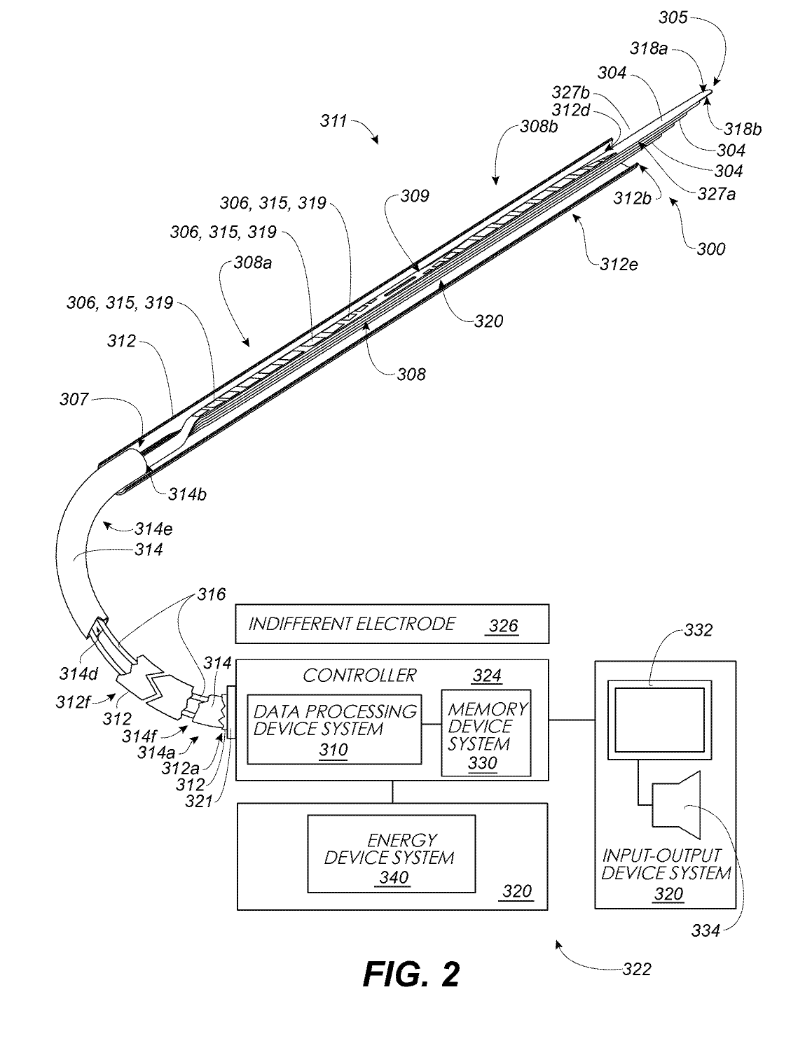

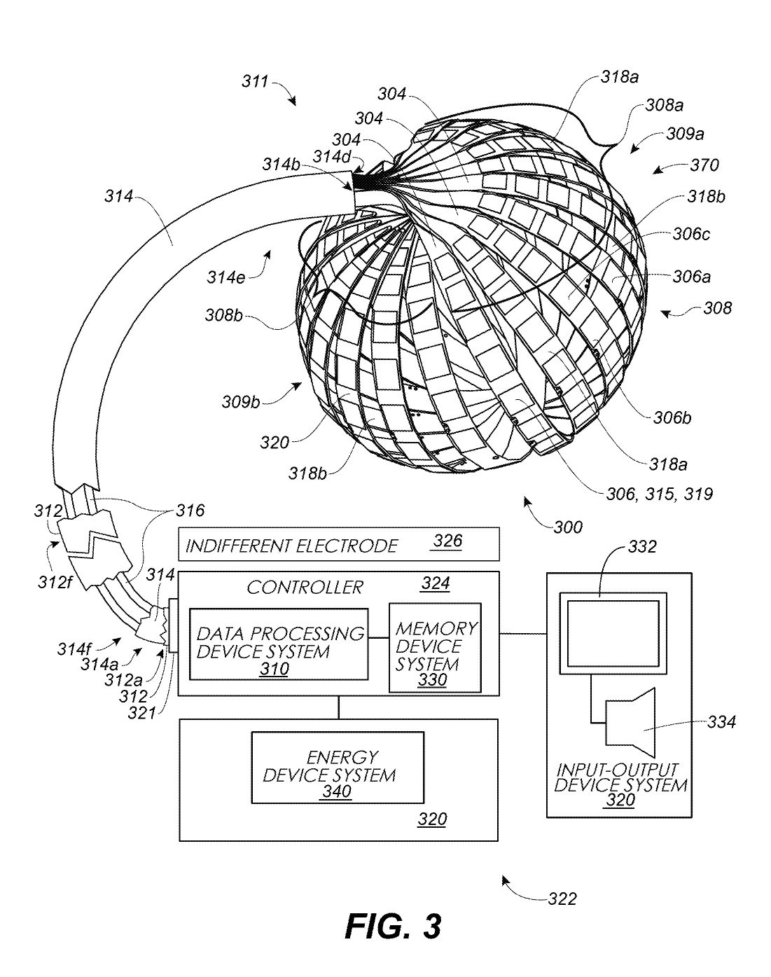

FIGS. 2 and 3 show a catheter system (i.e., a portion thereof shown schematically) that includes a catheter 311, according to some embodiments. The catheter 311 may correspond to catheter 206 and, in this regard, references herein to catheter 311 may be replaced with catheter 206, and vice versa, according to various embodiments. The catheter system of FIGS. 2 and 3 include a transducer-based device (also referred to as a manipulable portion) 300, according to some embodiments. The transducer-based device 300 may correspond to the transducer-based device 200 and, in this regard, may also be referred to as a manipulable portion, due to its ability to have its size, shape, or both size and shape altered, according to some embodiments. In this regard, references herein to transducer-based device 300 may be replaced with transducer-based device 200, and vice versa, according to various embodiments.

The catheter 311 may include an elongate shaft member that includes a proximal end, a distal end, and a length from the proximal end to the distal end. In this regard, the elongate shaft member may form part of or be a catheter sheath, such as catheter sheath 312 including proximal end 312a and distal end 312b. As another example, the elongate shaft member may form part of a catheter, such as elongate shaft member 314 of catheter 311. In various embodiments, the elongate shaft member 314 includes a first lumen 314d. The first lumen 314d may include a first end at least proximate a proximal end 314a of the elongate shaft member 314 and may include a second end at least proximate a distal end 314b of the elongate shaft member 314. Similarly, the catheter sheath 312 may include a first lumen 312d including a first end located at least proximate the proximal end 312a and may include a second end located at least proximate the distal end 312b of elongate shaft member 312. In this regard, various lumens may be provided in elongate shaft member 314, elongate shaft member 312, or both, to provide a passageway for various control leads (e.g., control leads 316) that may extend therethrough to various elongate members 304 or transducers 306 thereof that may form part of manipulable portion 300. Various lumens may be additionally or alternatively provided in elongate shaft member 314, elongate shaft member 312, or both, to provide a passageway for various control lines that may couple an actuator system (e.g., as known in the art, as described, e.g., in U.S. Pat. No. 9,452,016 (Moisa et al.), issued Sep. 27, 2016, which is hereby incorporated herein by reference in its entirety) to the manipulable portion 300 to selectively manipulate the manipulable portion 300 (e.g., selectively manipulating the manipulable portion 300 between an unexpanded or delivery configuration and an expanded or deployed configuration). In various embodiments, the distal end (e.g., 312b or 314b) of the elongate shaft member is arranged to be deliverable ahead of the proximal end (e.g., 312a or 314a) of the elongate shaft member through a bodily opening leading to a bodily cavity or a bodily organ. In some embodiments, the manipulable portion 300 is located at the distal end 314b of the elongate shaft member 314 or is located closer to the distal end 314b of the elongate shaft member 314 than to the proximal end 314a of the elongate shaft member 314. In some embodiments, the manipulable portion 300 is not located between the distal end 314b of the elongate shaft member 314 and the proximal end 314a of the elongate shaft member 314. In some embodiments, the length of the elongate shaft member (e.g., 312 or 314) is sufficient to position the proximal end (e.g., 312a or 314a) of the elongate shaft member outside a body that includes the bodily cavity during a state in which the distal end (e.g., 312b or 314b) of the elongate shaft member is positioned in the bodily cavity. In some embodiments, the length of the elongate shaft member (e.g., 312 or 314) is sufficient to position the proximal end (e.g., 312a or 314a) of the elongate shaft member outside a body that includes the bodily cavity during a state in which the manipulable portion is positioned at a desired location in the bodily cavity.

Transducer-based device 300 may include a plurality of elongate members 304 (three called out in each of FIGS. 2 and 3) and a plurality of transducers 306 (three called out in FIG. 2, and three called out in FIG. 3 as 306a, 306b, and 306c). As will become apparent, the plurality of transducers 306 is positionable within a bodily cavity. For example, in some embodiments, the transducers 306 can be positioned in a bodily cavity by movement into, within, or into and within the bodily cavity, with or without a change in a particular configuration of the plurality of transducers 306. In some embodiments, the plurality of transducers 306 are arrangeable to form a two- or three-dimensional distribution, grid or array of the transducers capable of mapping, ablating, or stimulating an inside surface of a bodily cavity or lumen without requiring mechanical scanning. As shown, for example, in FIG. 2, the plurality of transducers 306 are arranged in a distribution receivable in a bodily cavity, as the transducer-based device 300 and its plurality of transducers 306 are located within the catheter sheath 312 (also referred to as an elongate shaft member). Stated differently, in FIG. 2, for example, the plurality of transducers 306 are arranged in a distribution suitable to be deliverable to a bodily cavity. (It should also be noted, however, that the expanded or deployed configuration (e.g., FIGS. 1 and 3) may also be considered to have the transducers 306 arranged in a distribution receivable in a bodily cavity, as the transducer-based device 300 and its transducers 306 may be returned to the delivery configuration of FIG. 2, for example). In some embodiments, each of the transducers 306 includes an electrode 315 (one called out in FIG. 3) having an energy transmission surface 319 (one called out in FIG. 3) suitable for transmitting energy in various directions. In some embodiments, tissue-ablating energy is transmitted toward or away from an electrode 315. In some embodiments, tissue-based electrophysiological energy is transmitted toward an electrode 315.

The elongate members 304 may form part of a manipulable portion (e.g., 300), and in various embodiments, may form a frame or structure 308, the manipulable portion 300 and frame or structure 308 selectively moveable between an unexpanded or delivery configuration (i.e., as shown in FIG. 2) and an expanded or deployed configuration (i.e., as shown in FIG. 3) that may be used to reposition elongate members 304 in a particular desired arrangement. In this regard, it may also be stated that the transducer-based device, or manipulable portion, 300 is selectively moveable between an unexpanded or delivery configuration (i.e., as shown in FIG. 2) and an expanded or deployed configuration (i.e., as shown in FIG. 3). In some embodiments, the transducer-based device, or manipulable portion, 300, (e.g., the structure 308 thereof) has a size, shape, or both a size and a shape in the unexpanded or delivery configuration suitable to be percutaneously or intravascularly deliverable through a bodily opening (e.g., via an elongate shaft member such as catheter sheath 312, not shown in FIG. 3) to the bodily cavity. In some embodiments, structure 308 has a size, shape, or both a size and a shape in the expanded or deployed configuration too large to be percutaneously or intravascularly deliverable through a bodily opening (i.e., via catheter sheath 312) to the bodily cavity. The elongate members 304 may form part of a flexible circuit structure (i.e., also known as a flexible printed circuit board (PCB)). The elongate members 304 may include a plurality of different material layers, and each of the elongate members 304 may include a plurality of different material layers. The structure 308 may include a shape memory material, for instance Nitinol. The structure 308 may include a metallic material, for instance stainless steel, or non-metallic material, for instance polyimide, or both a metallic and non-metallic material by way of non-limiting example. The incorporation of a specific material into structure 308 may be motivated by various factors including the specific requirements of each of the unexpanded or delivery configuration and expanded or deployed configuration, the required position or orientation (i.e., pose) or both of structure 308 in the bodily cavity, or the requirements for successful ablation of a desired pattern. The number of elongate members depicted in FIG. 3 is non-limiting. The structure 308 may correspond to structure 218, and, in this regard, references herein to structure 308 may be replaced with structure 218, and vice versa, according to various embodiments.

Referring to FIGS. 2 and 3, transducer-based device or manipulable portion 300 may communicate with, receive power from, or be controlled by a control system 322. In some embodiments, elongate members 304 may form a portion of control leads or electrical conductors 316, for example, by stacking multiple layers, and terminating at a connector 321 or other interface with control system 322. The control leads 316 may correspond to the electrical connectors 216 in FIG. 1 in some embodiments. The control system 322 may include a controller 324 that may include a data processing device system 310 and a memory device system 330 that stores data and instructions that are executable by the data processing device system 310 to process information received from transducer-based device 300 or to control operation of transducer-based device 300, for example, activating various selected transducers 306 to ablate tissue. Controller 324 may include one or more controllers.

In some embodiments, the controller 324 may be configured to control deployment, expansion, retraction, or other manipulations of the shape, positioning, or both shape and positioning of the transducer-based device (e.g., manipulable portion) 300 at least by driving (e.g., by an electric or other motor) movement of various actuators or other catheter system components. In this regard, in some embodiments, the controller 324 is at least part of a control system, which may include one or more actuators, configured to advance at least part of the transducer-based device (e.g., 200 or 300), at least a portion of which may be considered a manipulable portion, out of the catheter sheath 312, retract at least part of the transducer-based device back into the catheter sheath 312, expand, contract, or otherwise change at least part of the shape of the transducer-based device.

Control system 322 may include an input-output device system 320 communicatively connected to the data processing device system 310 (i.e., via controller 324 in some embodiments). Input-output device system 320 may include a user-activatable control that is responsive to a user action. Input-output device system 320 may include one or more user interfaces or input/output (I/O) devices, for example one or more display device systems 332, speaker device systems 334, keyboards, mice, joysticks, track pads, touch screens or other devices to transfer information to, from, or both to and from a user, for example a care provider such as a health care provider or technician. For example, output from a mapping process may be displayed on a display device system 332.

Control system 322 may also include an energy device system 340 including one or more energy devices connected to transducers 306. In this regard, although FIG. 2 shows a communicative connection between the energy device system 340 and the controller 324 (and its data processing device system 310), the energy device system 340 may also be connected to the transducers 306 via a communicative connection that is independent of the communicative connection with the controller 324 (and its data processing device system 310). For example, the energy device system 340 may receive control signals via the communicative connection with the controller 324 (and its data processing device system 310), and, in response to such control signals, deliver energy to, receive energy from, or both deliver energy to and receive energy from one or more of the transducers 306 via a communicative connection with such transducers 306 (e.g., via one or more communication lines through elongate shaft member 314, control leads 316 or catheter sheath 312) that does not pass through the controller 324. In this regard, the energy device system 340 may provide results of its delivering energy to, receiving energy from, or both delivering energy to and receiving energy from one or more of the transducers 306 to the controller 324 (and its data processing device system 310) via the communicative connection between the energy device system 340 and the controller 324.

The energy device system 340 may, for example, be connected to various selected transducers 306 to selectively provide energy in the form of electrical current or power (e.g., RF energy), light or low temperature fluid to the various selected transducers 306 to cause ablation of tissue. The energy device system 340 may, for example, selectively provide energy in the form of electrical current to various selected transducers 306 and measure a temperature characteristic, an electrical characteristic, or both at a respective location at least proximate each of the various transducers 306. The energy device system 340 may include as its energy devices various electrical current sources or electrical power sources. In some embodiments, an indifferent electrode 326 is provided to receive at least a portion of the energy transmitted by at least some of the transducers 306. Consequently, although not shown in FIG. 2, the indifferent electrode 326 may be communicatively connected to the energy device system 340 via one or more communication lines in some embodiments. In addition, although shown separately in FIG. 2, indifferent electrode 326 may be considered part of the energy device system 340 in some embodiments. In some embodiments, the indifferent electrode 326 is provided outside the body or at least the bodily cavity in which the transducer-based device (e.g., 200 or 300) or catheter system is, at least in part, located.

In some embodiments, the energy device system 340 may include one or more driving motors configured to drive movement, in response to instructions from the controller 324, of various actuators or other catheter system components to control deployment, expansion, retraction, or other manipulations of the shape, positioning, or both shape and positioning of the transducer-based device (e.g., manipulable portion) 300.

It is understood that input-output device system 320 may include other systems. In some embodiments, input-output device system 320 may optionally include energy device system 340, transducer-based device 300 or both energy device system 340 and transducer-based device 300 by way of non-limiting example.

Structure 308 of transducer-based device 300 may be delivered and retrieved through at least a portion of a catheter member, for example, a catheter sheath 312. In some embodiments, the structure 308 provides expansion and contraction capabilities for a portion of a medical device (e.g., an arrangement, distribution or array of transducers 306). The transducers 306 may form part of, be positioned or located on, affixed to, mounted or otherwise carried on the structure, and the structure may be configurable to be appropriately sized to slide within a lumen of catheter sheath 312 in order to be deployed percutaneously or intravascularly. FIG. 2 shows one embodiment of such a structure.

In some embodiments, each of the elongate members 304 includes a respective distal end 305 (only one called out), a respective proximal end 307 (only one called out) and an intermediate portion 309 (only one called out) positioned between the proximal end 307 and the distal end 305. The respective intermediate portion 309 of each elongate member 304 includes a first or front surface 318a that is positionable to face an interior tissue surface within a bodily cavity and a second or back surface 318b opposite across a thickness of the intermediate portion 309 from the front surface 318a. In various embodiments, the intermediate portion 309 of each of the elongate members 304 includes a respective pair of side edges of the front surface 318a, the back surface 318b, or both the front surface 318a and the back surface 318b, the side edges of each pair of side edges opposite to one another, the side edges of each pair of side edges extending between the proximal end 307 and the distal end 305 of the respective elongate member 304. In some embodiments, each pair of side edges includes a first side edge 327a (only one called out in FIG. 2) and a second side edge 327b (only one called out in FIG. 2). In some embodiments, each of the elongate members 304, including each respective intermediate portion 309, is arranged front surface 318a-toward-back surface 318b in a stacked array during an unexpanded or delivery configuration (e.g., FIG. 2). In many cases, a stacked array allows the structure 308 to have a suitable size to be percutaneously or intravascularly deliverable. A stacked array can allow structure 308 to have a spatially efficient size for delivery through a lumen of catheter sheath 312. In some embodiments, the elongate members 304 are arranged to be introduced into a bodily cavity distal end 305 first. For clarity, not all of the elongate members 304 of structure 308 are shown in FIG. 2. A flexible catheter body or elongate shaft member 314 is used to deliver structure 308 through catheter sheath 312. In some embodiments, each elongate member includes a twisted portion proximate proximal end 307.

In some embodiments, each of the elongate members 304 is arranged in a fanned arrangement 370 in FIG. 3. In some embodiments, the fanned arrangement 370 is formed during the expanded or deployed configuration in which the transducer-based device (e.g., manipulable portion) 300 or structure 308 thereof is manipulated to have a size, shape, or both size and shape larger than in the delivery configuration, for example a size, shape, or both size and shape too large for percutaneous or intravascular delivery toward a bodily cavity, or a size, shape, or both size and shape too large for percutaneous or intravascular delivery away from a bodily cavity. In some embodiments, the fanned arrangement 370 is formed during the expanded or deployed configuration in which the transducer-based device (e.g., manipulable portion) 300 or structure 308 thereof is manipulated to have a size, shape, or both size and shape too large to be deliverable through a lumen of catheter sheath 312, for example, a size, shape, or both size and shape too large to be deliverable through a lumen of catheter sheath 312 toward a bodily cavity, or a size, shape, or both size and shape too large to be deliverable through a lumen of catheter sheath 312 away from a bodily cavity.

In some embodiments, the transducer-based device (e.g., manipulable portion) 300 or structure 308 thereof includes a proximal portion 308a having a first domed shape 309a and a distal portion 308b having a second domed shape 309b when the transducer-based device (e.g., manipulable portion) 300 or structure 308 thereof is in the expanded or deployed configuration. In some embodiments, the proximal and the distal portions 308a and 308b include respective portions of elongate members 304. In some embodiments, the transducer-based device (e.g., manipulable portion) 300 or structure 308 thereof is arranged to be delivered or advanced distal portion 308b first (e.g., distal portion 308b ahead of proximal portion 308a) into a bodily cavity when the transducer-based device (e.g., manipulable portion) 300 or structure 308 thereof is in the unexpanded or delivery configuration as shown in FIG. 2. In various example embodiments, each of the front surfaces 318a (two called out in FIG. 3) of the intermediate portions 309 of the plurality of elongate members 304 face outwardly from the structure 308 when the structure 308 is in the deployed configuration. In various example embodiments, each of the front surfaces 318a of the intermediate portions 309 of the plurality of elongate members 304 are positioned adjacent an interior tissue surface of a bodily cavity in which the structure 308 (i.e., in the deployed configuration) is located. In various example embodiments, each of the back surfaces 318b (two called out in FIG. 3) of the intermediate portions 309 of the plurality of elongate members 304 face inwardly toward an interior of the structure 308 when the structure 308 is in the deployed configuration.

The transducers 306 may be arranged in various distributions or arrangements in various embodiments. In some embodiments, various ones of the transducers 306 are spaced apart from one another in a spaced-apart distribution in the delivery configuration shown in FIG. 2. In some embodiments, various ones of the transducers 306 are arranged in a spaced-apart distribution in the deployed configuration shown in FIG. 3. In some embodiments, various pairs of transducers 306 are spaced apart with respect to one another. In other example embodiments, other structures may be employed to support or carry transducers of a transducer-based device such as a transducer-based catheter device. For example, an elongated catheter member may be used to distribute the transducers in a linear or curvilinear array. Basket catheters or balloon catheters may be used to distribute the transducers in a two-dimensional or three-dimensional array.

In some embodiments, a manipulable portion, such as, but not limited to, a transducer-based device (e.g., 200 or 300), is manipulated to transition between an unexpanded or delivery configuration (e.g., FIG. 2) and an expanded or deployed configuration (e.g., FIG. 3) manually (e.g., by a user's manual operation) or at least in part by way of motor-based driving (e.g., from the energy device system 340) of one or more actuators. Motor-based driving may augment or otherwise be in response to manual actions, may be responsive to automated control of a data processing device system (e.g., 310 in FIGS. 2 and 3), or may use a hybrid manual-automated approach.

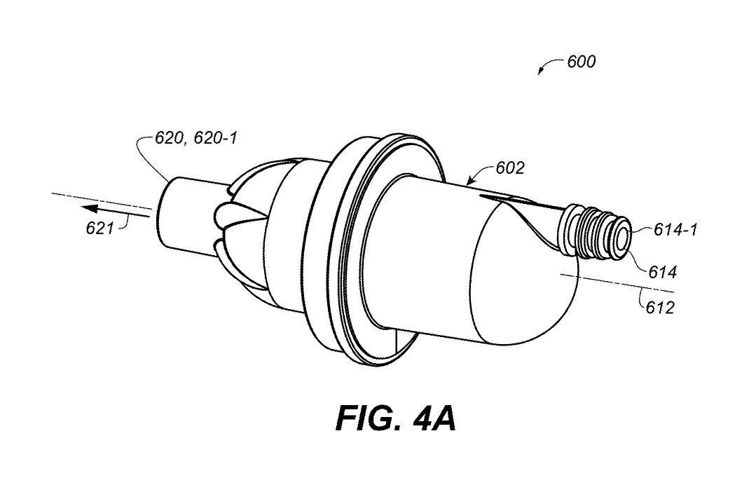

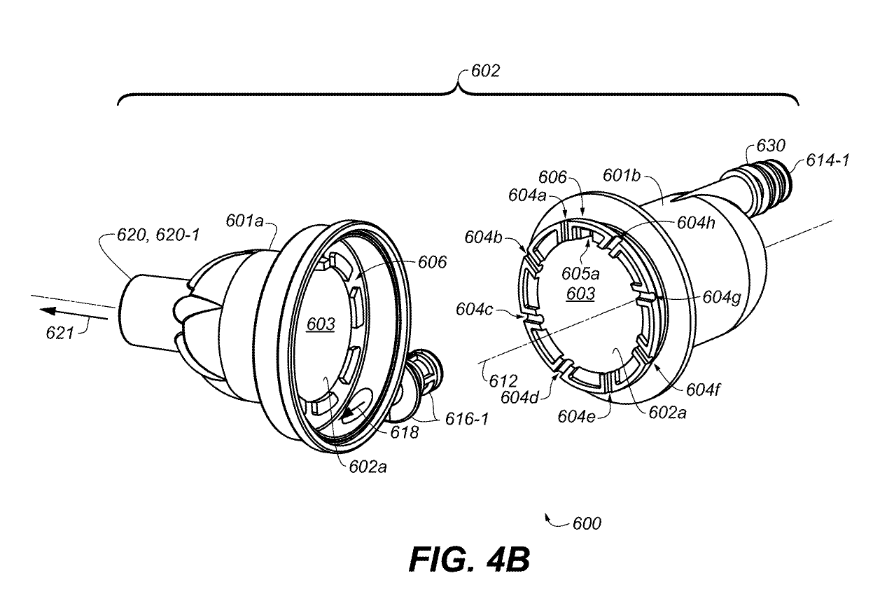

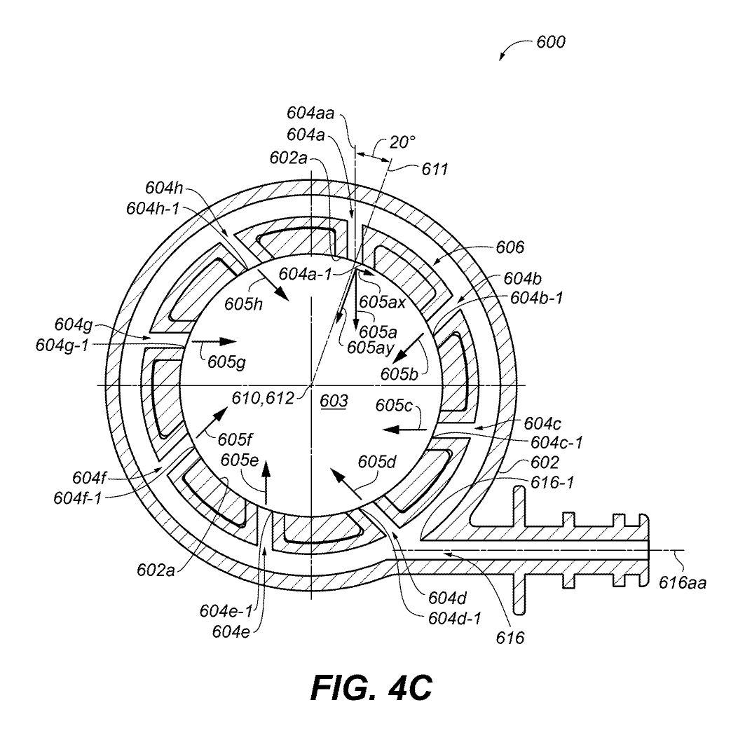

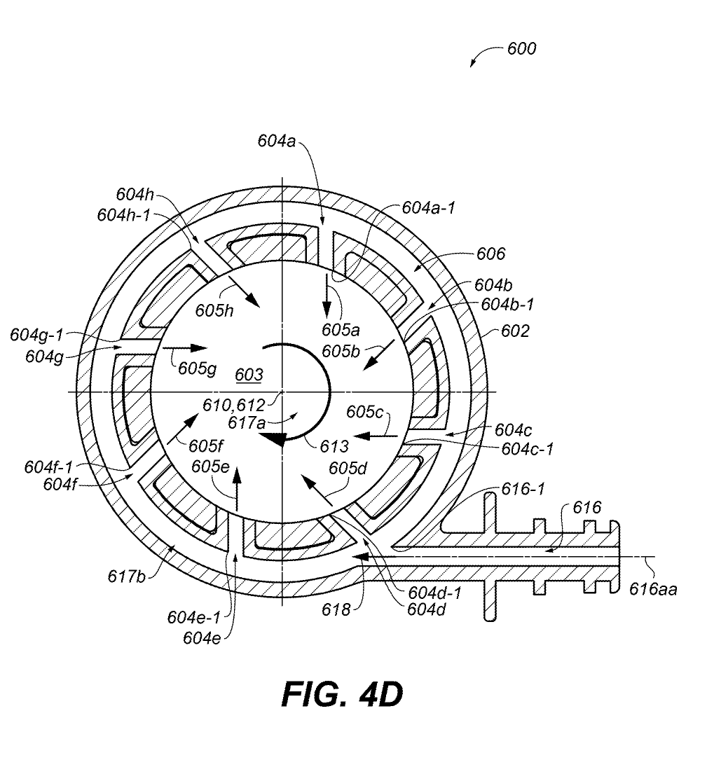

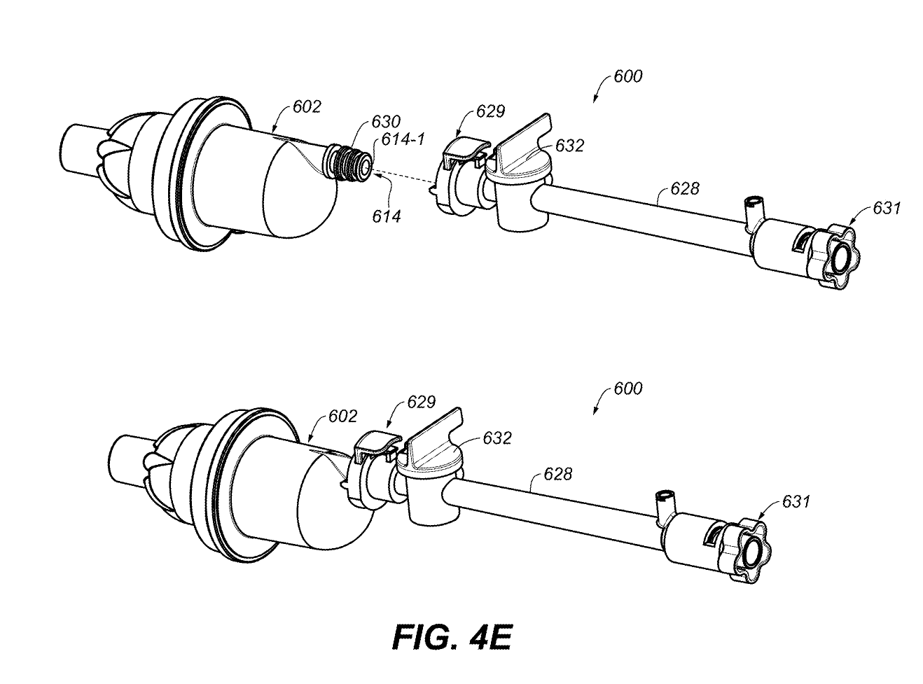

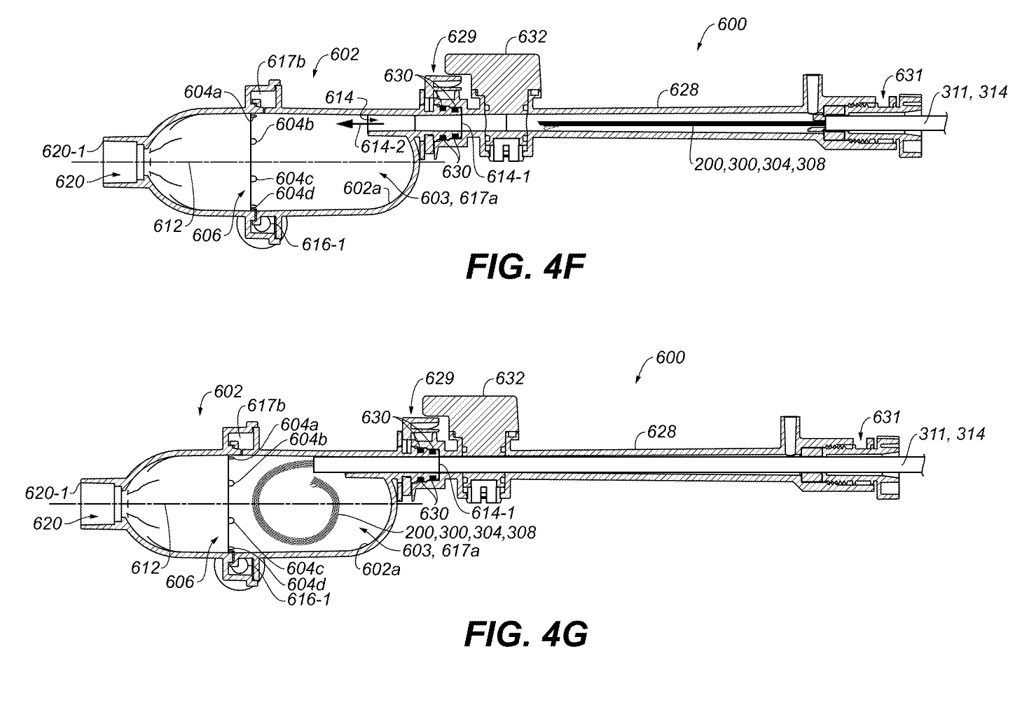

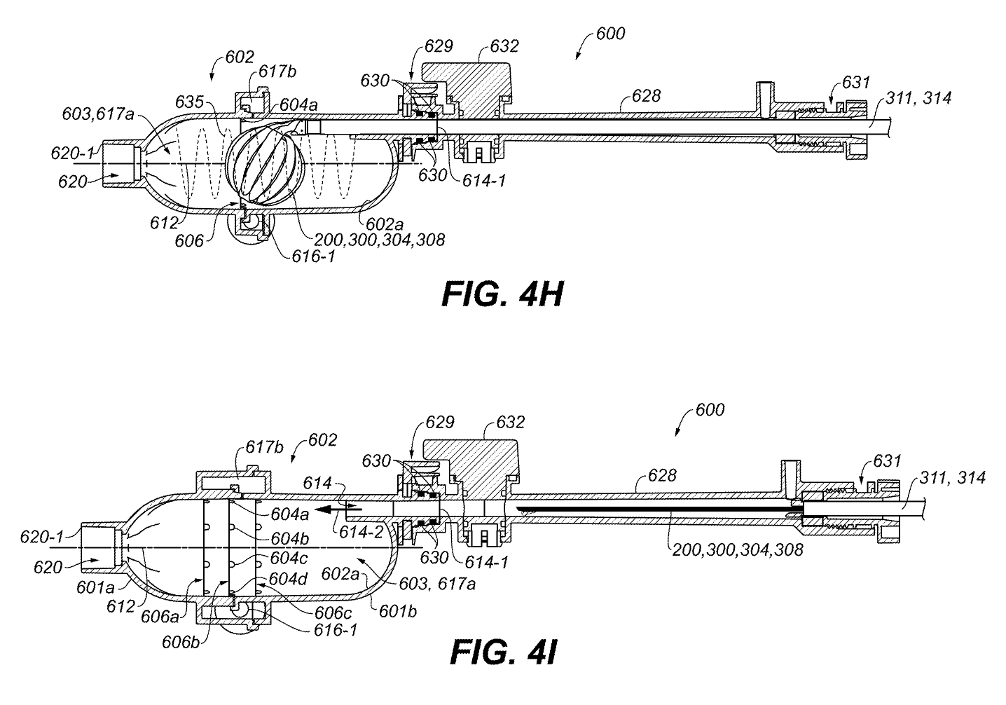

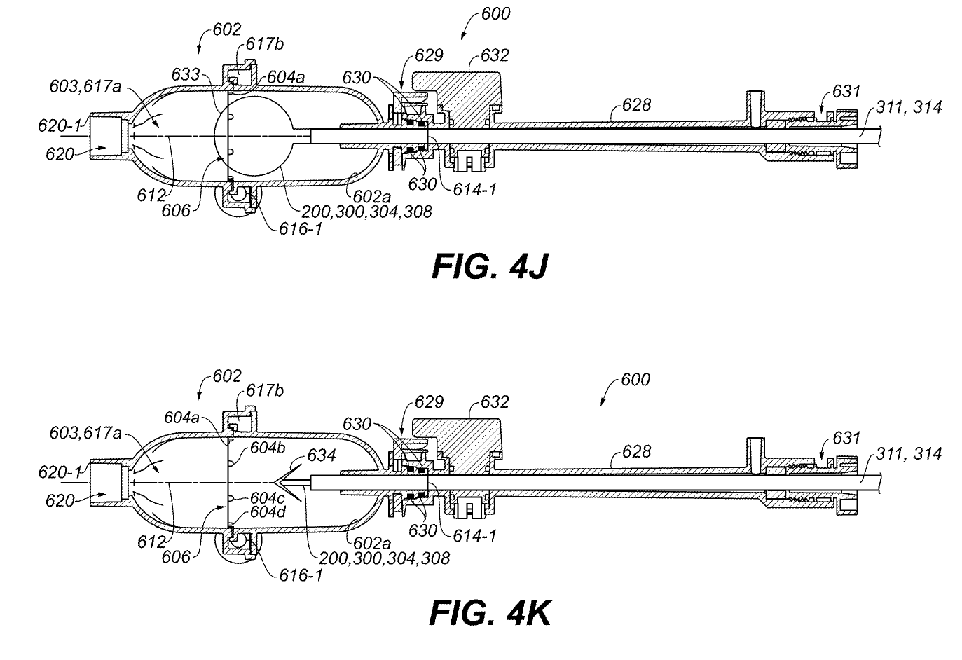

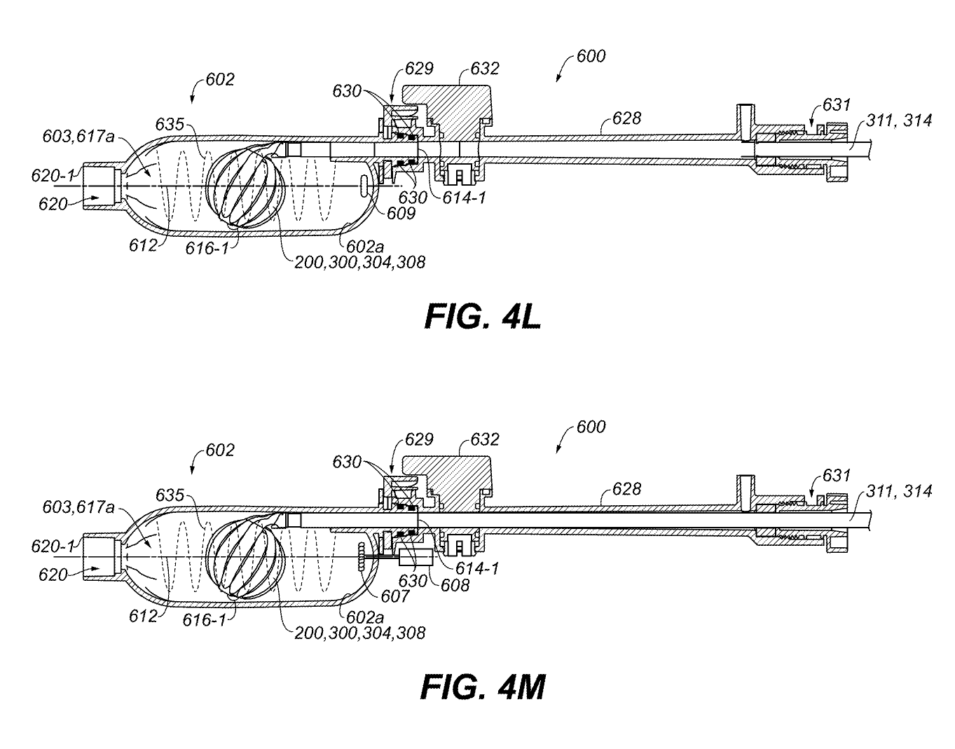

FIGS. 4A-4M (collectively referred to as FIG. 4) illustrate various aspects of medical device flushing apparatuses 600 employed to flush at least a portion of a medical device. In some embodiments, the medical device is a catheter, such as catheter 206 or 311, and the portion of the medical device is at least the manipulable portion 200 or 300 or respective structure 218 or 308 thereof, according to some embodiments.

According to various embodiments associated with at least FIGS. 4A, 4B, and 4C, flushing apparatus 600 may include an improved vessel or enclosure 602 that includes an interior surface 602a defining at least part of a flushing chamber including a flushing or internal cavity 603 (e.g., seen in FIG. 4B described below) configured to receive a structure (e.g., at least manipulable portion 300 or structure 308 thereof) of a medical device. According to various embodiments, the structure is a distal end structure of the medical device. For example, manipulable portion 300 or structure 308 may be considered distal end structures of catheter 311, because they are arranged or located on a distal portion (e.g., a distal portion 314e of the elongate shaft member 314 or a distal end portion 312e of the sheath 312) of the catheter 311, which is configured or arranged for insertion into a body of a patient, as opposed to a proximal portion (e.g., a proximal portion 314f of the elongate shaft member 314 or a proximal portion 312f of the sheath 312) of the catheter 311, which is configured or arranged to remain outside the body at least during a state in which the distal portion of the catheter 311 has been positioned at a desired location within the body.

According to some embodiments, the interior surface 602a includes a structure-receiving port 614-1 (seen in FIG. 4E) configured to receive the distal end structure (e.g., manipulable portion 300 or structure 308) of the medical device (e.g., catheter 311), at least part of the medical device configured to be percutaneously delivered toward a bodily cavity distal end structure first. In some embodiments, the structure-receiving port 614-1 may be also referred to as a medical device structure-receiving port 614-1. In some embodiments, the structure-receiving port is arranged to receive the structure or distal end structure (e.g., manipulable portion 300 or structure 308) of the medical device (e.g., catheter 311) to allow entry of at least a portion thereof into the flushing cavity (e.g., 603).

In some embodiments, the manipulable portion 300 and structure 308 are not configured to operate on or interact with tissue of a bodily cavity in a state in which the manipulable portion 300 and structure 308 are located within the enclosure 602 or any part of the flushing apparatus 600. In other words, the manipulable portion 300 and structure 308 are configured to operate on or interact with tissue of a bodily cavity in a state in which the manipulable portion 300 and structure 308 are not located within any part of the flushing apparatus 600 including the enclosure 602. For example, the elongate members 304 may include transducers, such as transducers 306 or transducers 220, which are configured to operate on or interact with tissue of a bodily cavity in a state in which the manipulable portion 300 and structure 308 are not located within any part of the flushing apparatus 600 including the enclosure 602. In a state in which the manipulable portion 300 and structure 308 are located within a part of the flushing apparatus 600, such as the enclosure 602, at least the part of the flushing apparatus 600 would prevent the transducers' operation on or interaction with tissue of a bodily cavity, according to various embodiments. In various embodiments, enclosure 602 is configured to not be deliverable, or configured to be incapable of being delivered, through the same bodily opening (e.g., via a percutaneous or intravascular delivery) that an elongate shaft member (e.g., 312 or 314) of the medical device system (or catheter device system) is configured to be deliverable through. In other words, there is no state of the enclosure 602 in which it is configured to be deliverable through the same bodily opening (e.g., via a percutaneous or intravascular delivery) that an elongate shaft member (e.g., 312 or 314) of the medical device system (or catheter device system) is configured to be deliverable through. For example, the enclosure 602 may include a size (e.g., an overall size or dimension) that is too large or renders the enclosure 602 too large for delivery of the enclosure 602 through the bodily opening leading to a bodily cavity to which the elongate shaft member (312 or 314) is to be delivered. For example, in FIGS. 4F, 4G, and 4H (described in further detail below), enclosure 602 is sized much larger than a size of the structure-receiving port 614-1, which is sized, according to various embodiments, to allow for a sliding fit with the at least part of the distal end portion (e.g., 312e or 314e) of the elongate shaft member (e.g., 312 or 314). Accordingly, the size of the structure-receiving port 614-1 may approximate the size (e.g., at least be smaller than the size) of the bodily opening through which the elongate shaft member (312 or 314) is to be delivered. Consequently, the enclosure 602 may be physically incapable of being delivered through such a bodily opening because its size is much larger than that of the structure-receiving port 614-1. In various embodiments, structure-receiving port 614-1 is provided by a structure-receiving channel 614.

In some embodiments, enclosure 602 may be incapable of being deliverable through the bodily opening in various states. For example, in some embodiments, the enclosure 602 may include a size (e.g., an overall size or dimension) that is too large or renders the enclosure 602 too large for delivery of the enclosure 602 through the bodily opening leading to a bodily cavity to which the elongate shaft member (e.g., 312 or 314) is to be delivered at least in a state in which a particular portion or the entirety of the internal cavity 603 is void of a particular liquid (e.g., a liquid such as a flushing liquid such as saline or heparinized saline). In some embodiments, the enclosure 602 may include a size (e.g., an overall size or dimension) that is too large or renders the enclosure 602 too large for delivery of the enclosure 602 through the bodily opening at least in a state in which a particular portion or the entirety of the internal cavity 603 is void of any particular liquid (e.g., void of any liquid whatsoever). In some embodiments, the enclosure 602 may include a size (e.g., an overall size or dimension) that is too large or renders the enclosure 602 too large for delivery of the enclosure 602 through the bodily opening at least in a state in which a portion of the internal cavity 603 in filled with a particular liquid and another portion of the internal cavity 603 is not filled with the particular liquid. Similarly, in some embodiments, the enclosure 602 may include a size (e.g., an overall size or dimension) that is too large or renders the enclosure 602 too large to fit in the bodily cavity to which the elongate shaft member (312 or 314) is to be delivered at least in each of one or more or all of the above-described states. For example, the enclosure 602 may have the above-described size(s) at least in embodiments where the enclosure 602 is formed of a rigid or substantially rigid structure that may be incapable of fitting into the bodily cavity or delivery though the bodily opening leading to the bodily cavity regardless of whether the enclosure 602 is empty of liquid or filled at least in part with liquid. FIG. 4A illustrates the enclosure 602 having such a rigid or substantially rigid structure. In some embodiments, enclosure 602 may include a structure that is incapable of collapsing to a size suitable for delivery through the bodily opening leading to the bodily cavity. For example, enclosure 602 may include a flexible structure that is collapsible or compressible to a minimum size that is incapable of allowing delivery of the enclosure 602 through the bodily opening leading to the bodily cavity.

Even if enclosure 602 is a flexible or substantially compliant structure that may allow sufficient compression to possibly fit through the bodily opening, the mere presence of the enclosure 602 during the delivery of the elongate shaft member (e.g., 312 or 314) through the bodily opening may impede, restrict, or prevent a required functioning of the elongate shaft member or catheter that comprises the elongate shaft member. For example, if the elongate shaft member is part of a catheter sheath (e.g., elongate shaft member 312), a delivery of an assembly including the elongate shaft member and enclosure 602 (i.e., positioned over the distal end portion (e.g., 312e) of the elongate shaft member) through the bodily opening would likely position the enclosure 602 so as to impede a subsequent delivery of the catheter or other medical instrument through a lumen of the elongate shaft member. If the elongate shaft member is part of a catheter (e.g., elongate shaft member 314), a delivery of an assembly including the elongate shaft member and enclosure 602 (i.e., positioned over the distal end portion (e.g., 314e) of the elongate shaft member) through the bodily opening would likely position the enclosure 602 so as to impede a subsequent operation of manipulable portion 300 with respect to tissue within the bodily cavity (e.g., tissue ablation or the sensing of various physiological parameters such as tissue electrical potential). In this regard, when the flushing apparatus 600 is employed, the at least part of the distal end portion (e.g., 312e or 314e) of the elongate shaft member (e.g., 312 or 314) inserted into the enclosure 602 is removed from the internal cavity 603 prior to a delivery of at least the distal end portion (e.g., 312e or 314e) of the elongate shaft member (e.g., 312 or 314) through the bodily opening leading to the bodily cavity, according to some embodiments.

According to various embodiments, the medical device flushing apparatus 600 includes at least one fluid-providing channel configured in an efficient and effective arrangement to consequently generate, at least in part, an improved fluid-flushing configuration. In some embodiments, the at least one fluid-providing channel interrupts the interior surface 602a of the enclosure 602. According to various embodiments, the at least one fluid-providing channel is configured to provide fluid to the flushing cavity 603. According to various embodiments, each of the at least one fluid-providing channel points, in a fluid-providing direction away from a centroid of the flushing cavity 603. For example, FIG. 4C is a two-dimensional cross-section of the flushing cavity 603 and its enclosure 602 of FIGS. 4A and 4B. According to various embodiments, the medical device flushing apparatus 600 includes at least one fluid-providing channel 604a that interrupts the interior surface 602a of the enclosure 602 and provides fluid to the flushing cavity 603, the at least one fluid-providing channel 604a pointing in a fluid-providing direction 605a that may obliquely enter the flushing cavity 603 and may point away from a centroid 610 of the flushing cavity 603 (the centroid 610 also being a centroid of the two-dimensional cross-section of the flushing cavity 603 shown in FIG. 4C, and the centroid 610 residing on a cross-sectional plane representing the plane of FIG. 4C). It is understood that the fluid-providing direction 605a as well as other fluid-providing directions (e.g., 605b, 605c, 605d, 605e, 605f, 605g, and 605h) described in this disclosure refers to a particular direction that fluid is provided or directed to or into the flushing cavity 603 by a respective fluid-providing channel. In FIG. 4C, the fluid-providing direction 605a associated with fluid-providing channel 604a, does not point toward centroid 610, but rather away from the centroid 610. In some embodiments, this orientation of the fluid-providing direction 605a may be associated with an effective flushing action provided by various embodiments.

According to some embodiments, the centroid 610 of the flushing cavity 603 is a point whose coordinates are the averages of the corresponding coordinates of a given set of points of a two-dimensional shape defined or bounded by the flushing cavity 603 (i.e., the centroid is provided by the geometric center of the two-dimensional shape). For example, in FIG. 4C, the cross-section of the flushing cavity 603 defines, according to some embodiments a circular two-dimensional shape, and the centroid 610 is provided by the geometric center of the circular shape. In such embodiments, the centroid 610 may be also referred to as a geometric centroid.

According to some embodiments, the centroid 610 of the flushing cavity 603 is a point whose coordinates are the averages of the corresponding coordinates of a given set of points of a three-dimensional volume defined or bounded by the flushing cavity 603. In some embodiments, the centroid of the flushing cavity 603 is provided by the center of gravity or the center of mass of the volume defined by, or bounded by, flushing cavity 603. In such embodiments, the centroid 610 may be also referred to as the center of mass or the center of gravity or alternatively as a volumetric centroid.

In some embodiments, both the at least one fluid-providing channel (e.g., 604a) and the centroid 610 are intersected by spatial plane (e.g., a spatial plane defined by the cross-section plane associated with FIG. 4C). In some embodiments, both the at least one fluid-providing channel (e.g., 604a) and the centroid 610 are intersected by a spatial plane (e.g., a spatial plane defined by the cross-sectional plane associated with FIG. 4C) and the at least one fluid-providing channel (e.g., 604a) is arranged such that its fluid-providing direction is parallel to the spatial plane, the at least one fluid-providing channel (e.g., 604a) pointing in the fluid-providing direction 605a away from the centroid 610. In some embodiments, both the at least one fluid-providing channel (e.g., 604a) and the centroid 610 are intersected by a spatial plane (e.g., a spatial plane defined by the cross-section plane associated with FIG. 4C) and the at least one fluid-providing channel (e.g., 604a) is arranged such that its longitudinal axis 604aa is parallel to or resides within the spatial plane, the at least one fluid-providing channel (e.g., 604a) pointing in the fluid-providing direction 605a away from the centroid 610.

In some embodiments, at least a portion of the flushing cavity 603 is defined by at least part of a body of revolution having an axis of revolution. For example, according to some embodiments, associated with FIG. 4, at least part of the interior surface 602a that defines flushing cavity 603 may form at least part of a body of revolution having an axis of revolution (e.g., axis 612). According to some embodiments, a body of revolution (e.g., sometimes also referred to as a solid of revolution) is a body obtained by rotating a planar figure about an axis in the same plane. According to some embodiments associated with FIG. 4, at least part of the flushing cavity 603 may have various portions (e.g., a cylindrical body portion or at least one hemispherical end portion) that may be defined as a body of revolution. It is noted that other shapes (e.g., ellipsoidal or conical) that may be provided by a body of revolution may form at least part of flushing cavity 603 in other embodiments. In some embodiments, axis 612 may also be considered to be a longitudinal axis of the flushing cavity 603. In some embodiments, the axis of revolution (e.g., axis 612) may extend through the centroid 610 of the flushing cavity 603 as exemplified in FIG. 4C. It is noted the axis 612 appears “on-end” in the view provided by FIG. 4C.

In some embodiments, the at least one fluid-providing channel (e.g., 604a) is arranged to obliquely interrupt the interior surface 602a of the enclosure 602. For example, as shown in FIG. 4C, the at least one fluid-providing channel (e.g., 604a) is not arranged to interrupt the interior surface 602a normally or orthogonally, but rather obliquely. This oblique interruption may be provided in some embodiments based on a particular positioning and orientation of the at least one fluid-providing channel (e.g., 604a), or a particular shape of the interrupted portion of the interior surface, or both. In some embodiments, this oblique interruption may be associated with an effective flushing action provided by various embodiments.

In some embodiments, the at least one fluid-providing channel (e.g., 604a) includes a plurality of fluid-providing channels. For example, as seen in FIG. 4C, the plurality of fluid-providing channels 604a, 604b, 604c, 604d, 604e, 604f, 604g, and 604h are provided, according to some embodiments. In some embodiments, each of the plurality of fluid-providing channels (e.g., 604a, 604b, 604c, 604d, 604e, 604f, 604g, and 604h) provide fluid to or into the flushing cavity 603. Each of the plurality of fluid-providing channels points in a fluid-providing direction (e.g., a respective one of fluid-providing directions 605a, 605b, 605c, 605d, 605e, 605f, 605g, and 605h), and each fluid-providing direction may obliquely enter the flushing cavity and may point away from the centroid 610 of the flushing cavity 603, according to some embodiments. In some embodiments, the fluid-providing channels of the plurality of fluid-providing channels (e.g., 604a, 604b, 604c, 604d, 604e, 604f, 604g, and 604h) are circumferentially arranged about the centroid 610 of the flushing cavity 603.

In some embodiments, the plurality of fluid-providing channels (e.g., 604a, 604b, 604c, 604d, 604e, 604f, 604g, and 604h) are aligned on a two-dimensional cross-section of the flushing cavity 603 (e.g., as shown in FIG. 4C). In some embodiments, a centroid of the two-dimensional cross-section of the flushing cavity 603 and the centroid 610 (e.g., a volumetric centroid) of the flushing cavity 603 are at a same location or, in some embodiments, appear at a same location at least as viewed along a line passing through the centroid of the two-dimensional cross-section, the line perpendicular to a plane in which the two-dimensional cross-section resides (e.g., as viewed in FIG. 4C, where the centroid of the two-dimensional cross-section of FIG. 4C may overlap or be overlapped by the centroid 610 of the flushing cavity 603 from these perspectives).

In some example embodiments, at least a portion of the flushing cavity 603 is defined by at least part of a body of revolution having an axis of revolution (e.g., axis 612), and the plurality of fluid-providing channels (e.g., 604a, 604b, 604c, 604d, 604e, 604f, 604g, and 604h) are circumferentially arranged about the axis of revolution (e.g., axis 612). In some embodiments, the axis of revolution (e.g., axis 612) extends through the centroid 610 of the flushing cavity (e.g., as exemplified in FIG. 4C). The collective nature and arrangement of the plurality of fluid-providing channels (e.g., 604a, 604b, 604c, 604d, 604e, 604f, 604g, and 604h), according to various embodiments, may provide an even further improved fluid-flushing action as compared to some contexts of embodiments that utilize a single fluid-providing channel. For example, as discussed below, a plurality of fluid-providing channels may more effectively generate a fluid vortex for flushing as compared to embodiments that utilize a single fluid-providing channel. However, in some contexts, provision of a single fluid-providing channel may reduce complexity and cost, and, therefore, may be preferable in some situations.

To further describe the effective orientation and arrangement of the fluid-providing channels, according to some embodiments, reference is made to characteristics of fluid-providing ports (e.g., ports 604a-1, 604b-1, 604c-1, 604d-1, 604e-1, 604f-1, 604g-1, and 604h-1) leading into the flushing cavity 603, the ports respectively formed by the plurality of fluid-providing channels (e.g., 604a, 604b, 604c, 604d, 604e, 604f, 604g, and 604h). According to various embodiments, each of the ports (e.g., 604a-1, 604b-1, 604c-1, 604d-1, 604e-1, 604f-1, 604g-1, and 604h-1) is located on the interior surface 602a. According to some embodiments, a first line (i.e., an imaginary straight line like an axis, not something structural) may extend through a centroid of the fluid-providing port along the fluid-providing direction of the fluid-providing channel. For example, in FIG. 4C, the first line may be provided by or represented by the longitudinal axis 604aa of fluid-providing channel 604a, the longitudinal axis 604aa extending, according to some embodiments, through a centroid of the port 604a-1 of the fluid-providing channel 604a. According to some embodiments, for each fluid-providing channel of the plurality of fluid-providing channels (e.g., 604a, 604b, 604c, 604d, 604e, 604f, 604g, and 604h), an angle between the first line and a second line (i.e., an imaginary straight line like an axis, not something structural) extending through the centroid of the fluid-providing port of the fluid-providing channel and the centroid 610 of the flushing cavity 603 is between 10 and 30 degrees. For example, in FIG. 4C, a second line 611 extends between the centroid of the fluid-providing port 604a-1 of the fluid-providing channel 604a and the centroid 610 of the flushing cavity 603, and an angle between the first line (e.g., represented by longitudinal axis 604aa) and the second line 611 is 20 degrees which lies in a range between 10 and 30 degrees. In some embodiments, the angle between the first line and the second line is between 10 and 60 degrees. In some embodiments, the angle is the same for at least two fluid-providing channels of the plurality of fluid-providing channels. These angles between 10 and 60 degrees and, in some contexts, beneficially between 10 and 30 degrees, may be associated with an effective flushing action provided by various embodiments. In some embodiments, the angle between the first line and the second line is as viewed along a third line passing through a centroid (e.g., 610) of a two-dimensional cross-section in which the plurality of fluid-providing channels are aligned (e.g., FIG. 4C), the third line perpendicular to a cross-sectional plane in which the two-dimensional cross-section resides (e.g., the view along the third line may be the perspective view of FIG. 4C). In other words, the angle between the first line and second line may be a projected angle projected onto the cross-sectional plane in which the two-dimensional cross-section resides.

In some embodiments, each fluid-providing direction (e.g., a respective one of fluid-providing directions 605a, 605b, 605c, 605d, 605e, 605f, 605g, and 605h) includes a directional component oriented to cause fluid rotation about the centroid of the flushing cavity. For example, as illustrated in FIG. 4C, fluid-providing direction 605a of fluid-providing channel 604a may be broken into an ‘x’ directional component 605ax and a ‘y’ directional component 605ay, where the ‘y-axis’ for the ‘y’ directional component 605ay is collinear with the second line 611, and the ‘x-axis’ for the ‘x’ directional component 605ax is perpendicular to the second line 611. In this example, the ‘x’ directional component 605ax is oriented to cause fluid rotation about the centroid 610 of the flushing cavity 603 in some embodiments.

Similarly, in some embodiments, the fluid-providing direction of each of at least one fluid-providing channel collectively is at least one fluid-providing direction such that the at least one fluid-providing direction is oriented to cause fluid rotation about the centroid of the flushing cavity. For example, with respect to FIG. 4C, a plurality of fluid-providing channels 604 are provided with their respective fluid-providing directions 605. In this regard, each fluid-providing direction 605 has a respective directional component like directional component 605ax, such that all of the fluid-providing directions 605 collectively are oriented to cause fluid rotation about the centroid 610 of the flushing cavity 603 in some embodiments.

In some embodiments, in which the plurality of fluid-providing channels (e.g., 604a, 604b, 604c, 604d, 604e, 604f, 604g, and 604h) are aligned on a two-dimensional cross-section of the flushing cavity 603 (e.g., as shown in FIG. 4C), a centroid of the two-dimensional cross-section of the flushing cavity 603 and the centroid 610 (e.g., a volumetric centroid) of the flushing cavity 603 may be at a same location or, in some embodiments, may appear at a same location at least as viewed along a line (e.g., the above-discussed third line) passing through the centroid of the two-dimensional cross-section, the line perpendicular to a plane in which the two-dimensional cross-section resides (e.g., as shown in FIG. 4C). In some embodiments, the two-dimensional cross-section resides on a cross-sectional plane (e.g., the spatial plane of FIG. 4C), and, for each fluid-providing channel of the plurality of fluid-providing channels (e.g., 604a, 604b, 604c, 604d, 604e, 604f, 604g, and 604h), the second line 611 extends along the cross-sectional plane through the centroid of the fluid-providing port of the fluid-providing channel and the centroid 610 of the flushing cavity 603.

According to some embodiments, the plurality of fluid-providing channels (e.g., 604a, 604b, 604c, 604d, 604e, 604f, 604g, and 604h) are circumferentially arranged about an axis (e.g., 612) extending through the centroid 610 of the flushing cavity 603, and the fluid-providing directions 605a, 605b, 605c, 605d, 605e, 605f, 605g, and 605h of the plurality of fluid-providing channels point in a same rotational direction about the axis (e.g., 612). For example, in the cross-section of the enclosure 602 in FIG. 4D, the fluid-providing directions 605a, 605b, 605c, 605d, 605e, 605f, 605g, and 605h of the plurality of fluid-providing channels (e.g., 604a, 604b, 604c, 604d, 604e, 604f, 604g, and 604h) point in a same rotational direction 613 (e.g., in a clockwise direction in this view) about the axis (e.g., 612) that the plurality of fluid-providing channels are circumferentially arranged about. In some embodiments, at least a portion of the flushing cavity 603 is defined by at least part of a body of revolution having an axis of revolution (e.g., 612) about which the plurality of fluid-providing channels (e.g., 604a, 604b, 604c, 604d, 604e, 604f, 604g, and 604h) are circumferentially arranged. According to various embodiments, each of the fluid-providing channels (e.g., 604a, 604b, 604c, 604d, 604e, 604f, 604g, and 604h) may point, in a fluid-providing direction (e.g., a respective one of 605a, 605b, 605c, 605d, 605e, 605f, 605g, and 605h), toward an interior of the flushing cavity 603, the fluid-providing directions of the plurality of fluid-providing channels pointing in a same rotational direction about the axis of revolution (e.g., 612).

The one or more fluid-providing channels (e.g., a plurality of fluid-providing channels, according to some embodiments associated with FIG. 4) may be provided or formed in the enclosure 602 in different manners. For example, as seen in FIG. 4B, in some embodiments, the enclosure 602 includes or is formed from a first housing 601a and a second housing 601b. Each of first housing 601a and second housing 601b may be made from various materials. Preferably, one or more materials that are capable of withstanding various sterilization processes required by various medical applications are employed. For example, in some embodiments, each of the first housing 601a and the second housing 601b is made from polycarbonate, one of various polymers known in the art that that can withstand the rigors of Ethylene oxide (EtO) sterilization. First housing 601a may be physically secured to the second housing 601b in various manners. For example, in some embodiments, the first housing 601a is bonded to the second housing 601b. In some embodiments, the first housing 601a is welded (e.g., ultrasonically welded) to the second housing 601b. In some embodiments, the first housing 601a is fastened (e.g., with (a) separate fasteners, or (b) integral fasteners (e.g., fasteners integrally provided in the first housing 601a, or the second housing 601b, or both the first and the second housings 601a and 601b), or both (a) and (b)) to the second housing 601b.

In some embodiments, each of at least a first fluid-providing channel of the one or more fluid-providing channels (e.g., 604a, 604b, 604c, 604d, 604e, 604f, 604g, and 604h) is defined or provided at least in part by each of the first housing 601a and the second housing 601b. For example, in FIG. 4B, the first housing 601a includes at least a first part of each fluid-providing channel, and the second housing includes at least a second part of each fluid-providing channel, with the first housing 601a physically couplable to the second housing 601b to form each fluid-providing channel in its entirety. Such an arrangement may be advantageous in particular embodiments in which the housings 601a, 601b are each molded as it simplifies the molds that are required to produce the housings. In some embodiments, at least a portion of the plurality of fluid-providing channels (e.g., 604a, 604b, 604c, 604d, 604e, 604f, 604g, and 604h) is provided in the first housing 601a, the second housing 601b, or each of both the first housing 601a and the second housing 601b. For example, in FIG. 4B, the first housing 601a includes at least a first part of each of all of the fluid-providing channels, and the second housing 601b includes at least a second part of each of all of the fluid-providing channels, each of the first housing 601a and the second housing 601b thereby including all of the plurality of fluid-providing channels. While one or more of the fluid-providing channels (e.g., 604a, 604b, 604c, 604d, 604e, 604f, 604g, and 604h) may be provided by both of the housings 601a, 601b, one or more of the fluid-providing channels may be provided entirely by one housing or the other, for instance, in embodiments where multiple rings 606 of fluid-providing channels are provided as shown, for example, in FIG. 4I (three rings 606a, 606b, and 606c shown in FIG. 4I for illustration purposes only), instead of the one ring 606 shown in at least FIGS. 4B, 4C, 4D, 4F, 4G, 4H, 4J, and 4K (note that the ring 606 in FIG. 4B is split across the two housings 601a, 601b). Such multiple rings may be a repetition of the structure shown in FIG. 4C in series in a stacked arrangement, for example, where the ring 606a of fluid-providing channels is entirely provided by the first housing 601a, the ring 606c of fluid-providing channels is entirely provided by the second housing 601b, and the ring 606b of fluid-providing channels is provided by the first housing 601a and the second housing 601b.

A description of some exemplary improved mechanisms, according to some embodiments, by which fluid may be provided to the one or more fluid-providing channels will now be provided. According to some embodiments, the enclosure 602 includes at least one fluid supply input port 616-1 (e.g., FIGS. 4C and 4D; also shown in FIGS. 4F, 4G, 4H, 4I, and 4J, but the fluid supply input port 616-1 is obscured behind the enclosure 612 in FIG. 4E). In some embodiments, the enclosure 602 fluidically couples the fluid supply input port 616-1 with the one or more fluid-providing channels (e.g., one or more or all of the plurality of fluid-providing channels 604a, 604b, 604c, 604d, 604e, 604f, 604g, and 604h in FIGS. 4C and 4D). In some embodiments, the flushing or internal cavity 603 is defined or provided at least in part by a first interior chamber 617a (FIG. 4D) of the enclosure 602. In some embodiments, the enclosure 602 includes a second interior chamber 617b (FIG. 4D), the second interior chamber 617b located at least in part between the first interior chamber 617a and the fluid supply input port 616-1. The first interior chamber 617a may be referred to as a flushing chamber in some embodiments. In some embodiments, the first interior chamber 617a may be referred to as defining at least part of a flushing chamber. For example, in some embodiments, the combination of the first interior chamber 617a and the second interior chamber 617b may be considered to form a flushing chamber. However, in some embodiments, for example, the first interior chamber 617a alone may be considered to define the flushing chamber. In some embodiments, the plurality of fluid-providing channels (e.g., 604a, 604b, 604c, 604d, 604e, 604f, 604g, and 604h) are located at least in part between the first interior chamber 617a and the second interior chamber 617b (e.g., as shown in FIG. 4D). In some embodiments, the second interior chamber 617b surrounds at least part of the first interior chamber 617a. In some embodiments, the structure-receiving port 614-1 is arranged to not interrupt an internal surface of the second interior chamber 617b (e.g., as shown in the cross-sections of FIGS. 4F, 4G, and 4H showing the structure-receiving port 614-1 distanced from the second interior chamber 617b). In some embodiments, a structure-receiving channel (e.g., 614) that provides the structure-receiving port 614-1 does not extend through the second interior chamber 617b (e.g., as shown in the cross-sections of FIGS. 4F, 4G, and 4H).

The use of the second interior chamber 617b, according to various embodiments, may be motivated by various reasons. For example, according to various embodiments, the second interior chamber 617b may be employed to directly supply fluid provided by the fluid supply input port 616-1 to all of the fluid-providing channels (e.g., 604a, 604b, 604c, 604d, 604e, 604f, 604g, and 604h) in embodiments in which the fluid-providing channels number greater than the number of fluid supply input ports.

In some embodiments (e.g., various embodiments associated with FIG. 4D), the fluid supply input port 616-1 is provided by a fluid input channel 616, and a longitudinal axis 616aa of the fluid input channel 616 extends toward the flushing cavity 603 along a particular direction that does not intersect the centroid 610 of the flushing cavity 603. In some embodiments, a fluid input channel (e.g., fluid input channel 616) providing the fluid supply input port 616-1 is arranged to cause fluid to enter an interior volume within the enclosure along a particular direction that does not intersect the centroid 610 of the flushing cavity 603. For example, in FIG. 4D, the fluid input channel 616 is configured or arranged to cause fluid to enter the second interior chamber 617b along fluid-providing direction 618. In some embodiments, each of at least some of the fluid-providing directions (e.g., 605a, 605b, 605d, 605e, 605f, 605g, and 605h) associated with the fluid provided into the flushing cavity 603 via respective ones of the fluid-providing channels (e.g., 604a, 604b, 604d, 604e, 604f, 604g, and 604h) is different than a particular direction (e.g., 618) in which a fluid input channel (e.g., 616) providing the fluid supply input port 616-1 is configured to cause fluid to enter an interior volume within enclosure 602. For example, in some embodiments according to FIG. 4D, all fluid-providing channels 604 except fluid-providing channel 604c is arranged to provide fluid in a fluid-providing direction 605 that is different than the particular direction 618 in which the fluid input channel 616 is configured to provide fluid into the interior volume within enclosure 602. In other words, fluid-providing direction 605c matches the particular direction 618 in FIG. 4D. However, in other embodiments, such as those that have a slight rotation of all of the fluid-providing channels 604 as compared to that shown in FIG. 4D, all of the fluid-providing channels 604 may be configured to provide fluid in fluid-providing directions 605 that are different than the particular direction 618 in which the fluid input channel 616 is configured to provide fluid into the interior volume within enclosure 602.

In some embodiments, the fluid input channel (e.g., 616) providing the fluid supply input port 616-1 is arranged to cause fluid to enter an interior volume within the enclosure along a particular direction (e.g., 618), and the particular direction and the respective fluid-providing directions (e.g., 605a, 605b, 605c, 605d, 605e, 605f, 605g, and 605h) associated with the plurality of fluid-providing channels are oriented in a same rotational direction (e.g., rotational direction 613 shown in FIG. 4D). In some embodiments, the plurality of fluid-providing channels (e.g., 604a, 604b, 604c, 604d, 604e, 604f, 604g, and 604h) are circumferentially arranged about an axis (e.g., 612), and a fluid input channel (e.g., 616) providing the fluid supply input port 616-1 is arranged to cause fluid to enter an interior volume within the enclosure along a particular direction (e.g., fluid-providing direction 618 along longitudinal axis 616aa of the fluid input channel 616) that has a directional component that is transversely oriented to a direction in which the axis (e.g., 612) extends. For example, in FIG. 4D, the fluid-providing direction 618 is transversely oriented to axis 612 which extends into and out of the page. According to some embodiments, the axis (e.g., 612) extends through the centroid 610 of the flushing cavity 603.

In some embodiments, at least a portion of the flushing cavity 603 is defined by at least part of a body of revolution having an axis of revolution (e.g., 612), and a fluid input channel (e.g., 616) providing the fluid supply input port 616-1 is arranged to cause fluid to enter an interior volume within the enclosure along a particular direction (e.g., 618) that has a directional component that is transversely oriented to a direction in which the axis of revolution (e.g., 612) extends. In some embodiments, the axis of revolution (e.g., 612) extends through the centroid 610 of the flushing cavity 603.

A description of some exemplary improved mechanisms, according to some embodiments, by which fluid may be removed from the flushing cavity 603 will now be provided. In some embodiments, the enclosure 602 includes a fluid output port 620-1 (e.g., FIGS. 4A, 4B, 4F, 4G, and 4H) fluidically coupled to the flushing cavity 603 to allow removal from the flushing cavity 603 of at least some of the fluid provided to the flushing cavity 603. In some embodiments, a fluid input channel (e.g., 616) providing the fluid supply input port 616-1 and a fluid output channel (e.g., 620) providing the fluid output port 620-1 are arranged such that a particular direction (e.g., 618) in which fluid enters an interior volume within the enclosure via the fluid input channel (e.g., 616) providing the fluid supply input port 616-1 and a particular direction (e.g., 621 and FIGS. 4A and 4B) in which fluid is removed from the flushing cavity 603 via the fluid output channel (e.g., 620) providing the fluid output port 620-1 are non-parallel directions (e.g., as exemplified in FIG. 4B). In some embodiments, at least a first fluid-providing channel (e.g., 604a) of the at least one fluid-providing channel and a fluid output channel (e.g., 620) providing the fluid output port 620-1 are arranged such that a particular direction (e.g., 605a) in which fluid enters the flushing cavity 603 via the first fluid-providing channel (e.g., 604a) and a particular direction (e.g., 621) in which fluid is removed from the flushing cavity 603 via the fluid output channel (e.g., 620) providing the fluid output port 620-1 are non-parallel directions (e.g., as exemplified in FIG. 4B).

In some embodiments, at least a portion of the plurality of fluid-providing channels (e.g., 604a, 604b, 604c, 604d, 604e, 604f, 604g, and 604h) are provided in the first housing 601a, the second housing 601b, or each of both the first housing 601a and the second housing 601b, and the fluid supply input port 616-1, the fluid output port 620-1, or each of the fluid supply input port 616-1 and the fluid output port 620-1 is provided, at least in part, by the first housing 601a or the second housing 601b, but not both the first housing 601a and the second housing 601b. For example, in FIG. 4B, each of the fluid supply input port 616-1 and the fluid output port 620-1 is provided by the first housing 601a, but not the second housing 601b.

Turning to FIG. 4E, a loading assembly 628 may be provided to assist insertion of a least part (e.g., manipulable portion 300 or structure 308) of the medical device into enclosure 602. In some embodiments, loading assembly 628 may be provided to assist insertion of a least part (e.g., manipulable portion 300 or structure 308) of the medical device into a catheter sheath (e.g., 312). In this regard, according to some embodiments, loading assembly 628 may be removably attached to enclosure 602 at least via coupler 629 located on a distal portion of the loading assembly 628. At least one seal 630 (e.g., an elastomeric seal) may be employed to reduce or eliminate potential fluid leaks between enclosure 602 and loading assembly 628. As described below with reference to FIGS. 4F, 4G, and 4H, the elongate shaft member (e.g., at least the elongate shaft member 314) may be inserted through a lumen of a loading assembly 628 to place a portion of the medical device (e.g., manipulable portion 300 or structure 308) into the internal cavity 603 of enclosure 602. A seal assembly 631 located, according to some embodiments, on a proximal portion of loading assembly 628 may be employed to reduce or eliminate potential fluid leaks at an interface between the loading assembly 628 and a portion of an elongate shaft member (e.g., 314) located in a lumen of the loading assembly 628. According to some embodiments, loading assembly 628 may include a valve 632 selectively operable to close a lumen of the loading assembly leading to the structure-receiving port 614-1 of enclosure 602. The use of the valve 632 may be motivated for various reasons. For example, according to some embodiments, after a portion of the medical device (e.g., manipulable portion 300 or structure 308) has been flushed (e.g., as per method 700 described below) in the flushing cavity 603 of the enclosure 602, the portion of the medical device may be retracted (e.g., via translation of elongate shaft member (e.g., 314) to which the portion of the medical device is physically coupled) into the loading assembly 628. The valve 632 may then be closed to maintain the flushed portion of the medical device submerged in flushing fluid in the loading assembly 628. The loading assembly 628 may then be physically coupled to the catheter sheath (e.g., 312) without unduly subjecting the portion of the medical device to a fluid (e.g., air) that could contaminate the portion of the medical device. Valve 632 would subsequently be opened to permit at least the portion of the medical device to be delivered through a lumen of the catheter sheath.

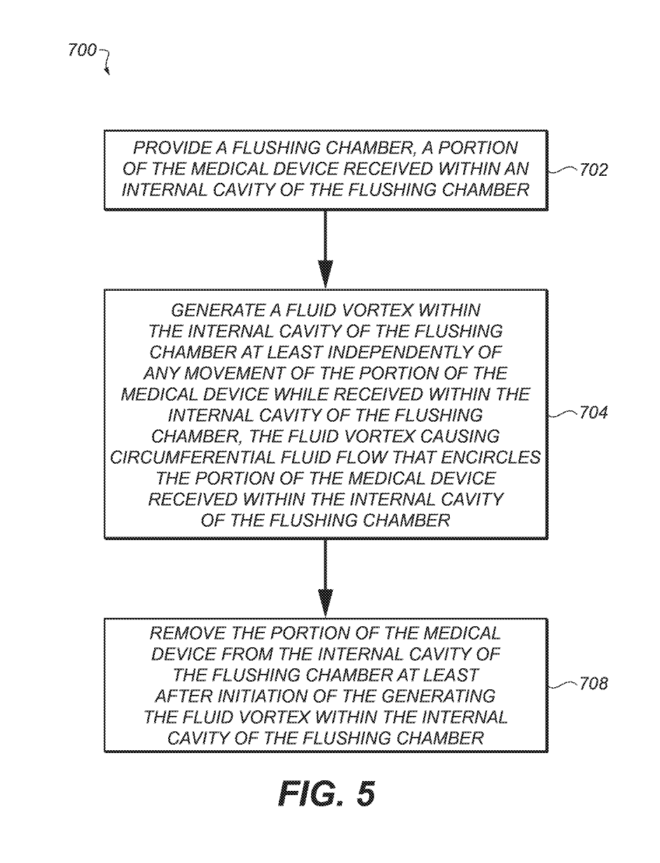

FIG. 5 is a block diagram representing a medical device flushing method 700 in which a portion of a medical device received in an internal cavity of a flushing chamber is flushed of an undesired fluid (e.g., air or other gases). For convenience of discussion, reference to flushing apparatus 600 may be employed to describe actions described by various blocks associated with method 700. It is understood, however, that method 700 or variants thereof may be employed with other types of flushing apparatuses, according to various embodiments. It is also understood that not all actions that make up method 700 are necessary in all embodiments, the invention is not limited to the particular ordering of actions described with respect to method 700, and various embodiments may include different sequences of actions.

In block 702, a flushing chamber is provided and a portion of the medical device is received within an internal cavity of the flushing chamber. For example, in some embodiments, at least part of the flushing apparatus 600 (e.g., enclosure 602 or a portion thereof such as the first interior chamber 617a or, e.g., both the first interior chamber 617a and the second interior chamber 617b) may provide the flushing chamber, and a portion of the medical device (e.g., manipulable portion 300 or structure 308) may be received in the internal cavity (e.g., flushing cavity 603). In some embodiments, the portion of the medical device is received in the internal cavity (e.g., flushing cavity 603) at a place of use of the medical device. In some embodiments, the portion of the medical device is received in the internal cavity (e.g., flushing cavity 603) at a location remote from a particular location in which a medical procedure employing the medical device is conducted. For example, the portion of the medical device may be received in the internal cavity (e.g., flushing cavity 603) at a location where the medical device is manufactured or a location where flushing chamber (e.g., 602) is manufactured. According to various embodiments, the portion of the medical device is received in the internal cavity (e.g., flushing cavity 603) prior to the portion of the medical device being inserted into the body of a patient. For instance, as discussed above, according to various embodiments, the manipulable portion 300 and structure 308 are not configured to operate on or interact with tissue of a bodily cavity in a state in which the manipulable portion 300 and structure 308 are located within the enclosure 602 or any part of the flushing apparatus 600. In other words, for example, the manipulable portion 300 and structure 308 are configured to operate on or interact with tissue of a bodily cavity in a state in which the manipulable portion 300 and structure 308 are not located within any part of the flushing apparatus 600 including the enclosure 602.

In some embodiments, the portion of the medical device is a distal end structure of a medical device (e.g., manipulable portion 300 or structure 308), at least part of the medical device configured to be percutaneously delivered toward a bodily cavity distal end structure first. In some embodiments, the portion of the medical device includes a structure (e.g., 218 or 308) including a plurality of transducers. In some embodiments, at least some of the transducers (e.g., 220 or 306) are operable to selectively emit tissue-ablative energy.

In some embodiments, the portion of the medical device includes a structure (e.g., 218 or 308) that is selectively moveable between a delivery configuration and a deployed configuration (examples of delivery and deployed configurations are discussed above). In some embodiments, the structure (e.g., 218 or 308) includes a plurality of elongate members (e.g., 304), a set of one or more transducers located on each of at least some of the plurality of elongate members. For example, as shown at least in FIG. 3, each elongate member 304 includes its own set of a plurality of transducers 306. In some embodiments, the structure includes an expandable balloon (e.g., a balloon catheter 633 shown for instance in FIG. 4J). In some embodiments, the portion of the medical device includes an implant (e.g., implant 634 schematically shown for instance in FIG. 4K) configured for implantation within the body of a patient.

In some embodiments in which the structure (e.g., manipulable portion 300 or structure 308) of the medical device is selectively moveable between a delivery configuration and a deployed configuration (examples of delivery and deployed configurations are discussed above), the medical device structure-receiving port 614-1 is sized to receive the structure of the medical device in a state in which the structure is in the delivery configuration, but not the deployed configuration. For example, FIGS. 4F, 4G, and 4H show sectioned views of enclosure 602 at three separate times during an insertion of catheter 311 into enclosure 602, according to some embodiments. It is noted that loading assembly 628 (i.e., shown in section in FIGS. 4F, 4G, and 4H) also is employed for the insertion process, according to some embodiments. In FIG. 4F, the manipulable portion 300 or structure 308 is shown in a delivery configuration in which the manipulable portion 300 or structure 308 (i.e., shown positioned in loading assembly 628) is arranged in a suitable configuration (e.g., a stacked configuration) suitably sized to be deliverable through the loading assembly 628 and suitably sized to be deliverable through the structure-receiving channel 614 and its associated structure-receiving port 614-1. In FIG. 4G, the manipulable portion 300 or structure 308 has been delivered into the flushing cavity 603 and has been moved into a first deployed configuration (e.g., a coiled configuration) which causes the manipulable portion 300 or structure 308 to have a size, according to some embodiments, too large to be deliverable through the loading assembly 628 and too large to be deliverable through the structure-receiving channel 614 and its associated structure-receiving port 614-1.

It is noted that the elongate members 304 that make up manipulable portion 300 or structure 308, according to various embodiments, are depicted schematically in FIG. 4G, and manipulable portion 300 or structure 308 may include other elongate members 304 that are not shown.

In FIG. 4H, the manipulable portion 300 or structure 308 has been delivered into the flushing cavity 603 and has been moved into a second deployed configuration (e.g., a fanned configuration), which, like the first deployed configuration, is a configuration in which the manipulable portion 300 or structure 308 has a size, according to some embodiments, too large to be deliverable through the loading assembly 628 and too large to be deliverable through the structure-receiving channel 614 and its associated structure-receiving port 614-1. In some embodiments, the manipulable portion 300 or structure 308 is moved into the fanned second deployed configuration from the coiled first deployed configuration. In some embodiments, the manipulable portion 300 or structure 308 is positioned in the deployed configuration in a state in which it is located within the flushing or internal cavity 603, as shown, e.g., in FIGS. 4G and 4H. U.S. Pat. No. 9,526,573, issued Dec. 27, 2016, which is hereby incorporated herein by reference in its entirety, describes various mechanisms and techniques by which a structure of a medical device may be manipulated and moved between various configurations.

In some embodiments, method 700 may include moving the structure (e.g., manipulable portion 300 or structure 308) between a delivery configuration and a deployed configuration within the internal cavity of the flushing chamber. In some embodiments, method 700 may include moving the structure (e.g., manipulable portion 300 or structure 308) (a) from a delivery configuration to a deployed configuration within the internal cavity of the flushing chamber, or (b) from a deployed configuration to a delivery configuration within the internal cavity of the flushing chamber, or both (a) and (b). According to some embodiments, expanding movements of the structure within the internal cavity of the flushing chamber may facilitate flushing of undesired fluid (e.g., air) from the structure during the flushing procedures described herein. In some embodiments, the medical device structure-receiving port 614-1 is provided by a medical device structure-receiving channel 614 that is positioned to constrain entry of the portion of the structure (e.g., manipulable portion 300 or structure 308) into the flushing cavity 603 along a direction 614-2 (shown, e.g., in FIG. 4F) that does not point toward the centroid 610 of the flushing cavity 603. For example, as shown by at least a combination of FIGS. 4F and 4D, the direction 614-2 points to a perimeter region of the flushing cavity 603, away from the centroid 610 of the flushing cavity 603 shown in FIG. 4D, according to some embodiments. The direction 614-2 would point into the plane of FIG. 4D toward the top of FIG. 4D. A fluid vortex may be generated within the internal cavity (e.g., flushing cavity 603) of the flushing chamber (e.g., at least the first interior chamber 617a of enclosure 602) as indicated by block 704 of method 700. According to some embodiments, the fluid vortex is generated within the internal cavity of the flushing chamber by causing fluid movement within the internal cavity of the flushing chamber at least independently of any movement of the portion of the medical device while the portion of the medical device is received within the internal cavity of the flushing chamber. That is, the fluid vortex is generated by an action that does not require moving or otherwise manipulating (e.g., whisking) the portion of the medical device while the portion of the medical device is received in the internal cavity of the flushing chamber, although movement or other manipulation (e.g., reciprocation, expansion, contraction, etc.) of the portion of the medical device within the generated fluid vortex may be desirable to enhance or expedite removal of undesired fluid (e.g., air) from the portion of the medical device. In other words, the portion of the medical device being flushed is not needed to generate the fluid vortex. According to some embodiments, the portion of the medical device includes a structure (e.g., manipulable portion 300 or structure 308) that is selectively moveable between a delivery configuration and a deployed configuration (examples of delivery and deployed configurations are discussed above), and the generating the fluid vortex within the internal cavity of the flushing chamber occurs at least independently of any movement of the portion of the medical device while the structure is received within the internal cavity of the flushing chamber in the deployed configuration.

It is noted that, according to various embodiments, the fluid vortex may be generated in the internal cavity of the flushing chamber at least in absence of the portion of the medical device being present in the internal cavity of the flushing chamber. That is, in some embodiments, the portion of the medical device need not be present in the internal cavity of the flushing chamber to allow the fluid vortex to be formed within the internal cavity.

In some embodiments, the structure (e.g., manipulable portion 300 or structure 308) is moved between the delivery configuration and the deployed configuration within the internal cavity of the flushing chamber at least before an initiation of the generating the fluid vortex within the internal cavity of the fluid chamber. For example, the structure (e.g., manipulable portion 300 or structure 308) may proceed toward the internal cavity (e.g., flushing cavity 603) of the flushing chamber (e.g., at least the first interior chamber 617a of the enclosure 602) in the delivery configuration (e.g., a delivery configuration as shown in FIG. 4F), and when at least a portion of the structure is in the internal cavity of the flushing chamber, it may be moved into a deployed configuration (e.g., a deployed configuration shown for example in FIG. 4G or 4H) in the internal cavity before (or in some embodiments, during or after) the generating of the fluid vortex. Such action of moving at least the portion of the structure into a deployed configuration prior to fluid vortex generation may help prepare for flushing of the at least the portion of the structure by expanding the at least the portion of the structure. According to various embodiments, the fluid vortex causes circumferential fluid flow that encircles the portion of the medical device received within the internal cavity of the flushing chamber to flush the portion of the medical device of undesired fluid (e.g., air), in a state in which the portion of the medical device is received within the internal cavity of the flushing chamber.

Generating the fluid vortex within the internal cavity (e.g., flushing cavity 603) of the flushing chamber (e.g., at least the first interior chamber 617a of the enclosure 602) as per block 704 may be accomplished in various manners, according to various embodiments. According to some embodiments, the method 700 includes injecting fluid (e.g., a flushing fluid like saline or heparinized saline) into the internal cavity (e.g., flushing cavity 603) of the flushing chamber (e.g., at least the first interior chamber 617a of the enclosure 602) to cause, at least in part, the generating the fluid vortex within the internal cavity (e.g., flushing cavity 603) of the flushing chamber (e.g., at least the first interior chamber 617a of the enclosure 602) as per block 702. For example, in some embodiments, the injecting fluid into the internal cavity (e.g., flushing cavity 603) of the flushing chamber (e.g., at least the first interior chamber 617a of the enclosure 602) includes injecting fluid from a fluid input channel (e.g., 616) providing a fluid supply input port (e.g., 616-1) into the internal cavity (e.g., flushing cavity 603) of the flushing chamber (e.g., at least the first interior chamber 617a of the enclosure 602) via at least one or all of the fluid-providing channels (e.g., 604a-604h) interrupting an interior surface (e.g., 602a) of the flushing chamber, each of the at least one fluid-providing channel (e.g., 604a) pointing, in a fluid-providing direction (e.g., 605a), toward the interior of the internal cavity (e.g., flushing cavity 603) of the flushing chamber (e.g., at least the first interior chamber 617a of the enclosure 602), each fluid-providing direction (e.g., 605a) pointing away from a centroid (e.g., 610) of the internal cavity (e.g., flushing cavity 603) of the flushing chamber (e.g., at least the first interior chamber 617a of the enclosure 602) as exemplified in FIG. 4C. In some embodiments, the injecting fluid into the internal cavity (e.g., flushing cavity 603) of the flushing chamber (e.g., at least the first interior chamber 617a of the enclosure 602) includes injecting fluid into the internal cavity (e.g., flushing cavity 603) of the flushing chamber (e.g., at least the first interior chamber 617a of the enclosure 602) via at least one or all of the fluid-providing channels (e.g., 604a-604h) which are arranged to obliquely interrupt an interior surface (e.g., 602a) of the enclosure (e.g., 602) as exemplified in FIG. 4C.

According to various embodiments, the at least one fluid-providing channel (e.g., 604a) is oriented to generate a fluid vortex within the internal cavity (e.g., flushing cavity 603) at least in a state in which fluid is provided to the internal cavity (e.g., flushing cavity 603). In some embodiments, the internal cavity (e.g., flushing cavity 603) is configured to cause the fluid vortex to spin around the centroid (e.g., 610) of the internal cavity (e.g., flushing cavity 603) when the fluid vortex is generated within the internal cavity (e.g., flushing cavity 603) at least in a state in which fluid is provided to the internal cavity (e.g., flushing cavity 603). For example, each fluid-providing channel of the at least one fluid-providing channel (e.g., 604a) may be oriented to (a) point, in a fluid-providing direction (e.g., 605a), toward the interior of the internal cavity (e.g., flushing cavity 603) of the flushing chamber (e.g., at least the first interior chamber 617a of the enclosure 602), each fluid-providing direction (e.g., 605a) pointing away from a centroid (e.g., 610) of the internal cavity (e.g., flushing cavity 603) of the flushing chamber (e.g., at least the first interior chamber 617a of the enclosure 602), or (b) obliquely interrupt an interior surface (e.g., 602a) of the flushing chamber (e.g., at least the first interior chamber 617a of the enclosure 602), or both (a) and (b) to cause fluid injected via the at least one fluid-providing channel (e.g., 604a) to generate the fluid vortex that spins around the centroid 610 of the internal cavity (e.g., flushing cavity 603). Similarly, in some embodiments where a plurality of fluid-providing channels 604 are aligned on a two-dimensional cross-section of the flushing cavity (e.g., as shown in at least FIG. 4C), the internal cavity (e.g., flushing cavity 603) is configured to cause the fluid vortex to spin around a centroid of the two-dimensional cross-section of the flushing cavity.

In some embodiments, when the one or more fluid-providing channels include a plurality of fluid-providing channels (e.g., 604a, 604b, 604c, 604d, 604e, 604f, 604g, and 604h), the plurality of fluid-providing channels may be collectively oriented to generate a fluid vortex within the internal cavity (e.g., flushing cavity 603) in a state in which fluid is provided to the internal cavity (e.g., flushing cavity 603). In some embodiments, the generated fluid vortex helically spins about an axis centrally located in the generated fluid vortex at least in the state in which fluid is provided to the internal cavity (e.g., flushing cavity 603). According to some embodiments, the fluid-providing directions (e.g., 605a, 605b, 605c, 605d, 605e, 605f, 605g, and 605h) of the plurality of fluid-providing channels point in a same rotational direction about the axis (e.g., rotational direction 613 about axis 612 in FIG. 4D). According to some embodiments, the vortex may spin about axis 612. In some embodiments, the flushing chamber (e.g., at least the first interior chamber 617a of the enclosure 602) includes a fluid supply input port (e.g., 616-1, FIGS. 4C, 4D, 4E, 4F, 4G, and 4H), and the flushing chamber enclosure (e.g., enclosure 602) fluidically couples (e.g., via second interior chamber 617b) the fluid supply input port to all of the plurality of fluid-providing channels (e.g., 604a, 604b, 604c, 604d, 604e, 604f, 604g, and 604h), with the fluid supply input port (e.g., 616-1) spaced from the axis (e.g., axis 612) about which the generated fluid vortex helically spins. In some embodiments, the plurality of fluid-providing channels (e.g., 604a, 604b, 604c, 604d, 604e, 604f, 604g, and 604h) are collectively oriented to generate a fluid vortex within the internal cavity (e.g., flushing cavity 603) in a state in which fluid is provided to the internal cavity, the generated fluid vortex helically spinning about an axis, and a channel (e.g., 616) fluidically coupled to the fluid supply input port is arranged to cause fluid to enter an interior volume within the enclosure containing flushing chamber (e.g., the fluid input channel 616 provides fluid into the second interior chamber 617b of the enclosure 602) along a particular direction that has a directional component that is transversely oriented to a direction in which the axis extends. In some embodiments, the plurality of fluid-providing channels (e.g., 604a, 604b, 604c, 604d, 604e, 604f, 604g, and 604h) is circumferentially arranged about the axis that the generated fluid vortex spins around. In some embodiments, at least a portion of the internal cavity (e.g., flushing cavity 603) is defined by at least part of a body of revolution having an axis of revolution, and the generated fluid vortex spins about the axis of revolution. In some embodiments, the axis that the generated fluid vortex spins about is collinear with axis 612. In some embodiments, the flushing chamber (e.g., at least the first interior chamber 617a of the enclosure 602) includes a medical device receiving port (e.g., the structure-receiving port 614-1) configured to receive the portion of the medical device and allow for a positioning of the portion of the medical device such that the circumferential flow caused by the fluid vortex encircles the portion of the medical device. In some embodiments, the medical device receiving port (e.g., the structure-receiving port 614-1) is spaced from the axis that the generated fluid vortex spins about.

In some embodiments, the causing fluid movement within the internal cavity of the flushing chamber as per block 704 includes providing fluid into the internal cavity (e.g., flushing cavity 603) via a fluid supply input port (e.g., 616-1), and an enclosure (e.g., 602) fluidically couples the fluid supply input port (e.g., 616-1) with all of the plurality of fluid-providing channels (e.g., 604a, 604b, 604c, 604d, 604e, 604f, 604g, and 604h), and a fluid input channel (e.g., 616) providing the fluid supply input port (e.g., 616-1) is arranged to cause fluid to enter an interior volume within the enclosure containing the flushing chamber (e.g., the fluid input channel 616 provides fluid into the second interior chamber 617b of the enclosure 602) along a particular direction that does not intersect a centroid (e.g., 610) of the internal cavity (e.g., flushing cavity 603) of the flushing chamber.

In some embodiments, the causing fluid movement within the internal cavity of the flushing chamber as per block 704 includes providing fluid into the internal cavity (e.g., flushing cavity 603) via at least a fluid input channel (e.g., 616) providing a fluid supply input port (e.g., 616-1), and via at least an enclosure (e.g., 602) that fluidically couples the fluid supply input port to the plurality of fluid-providing channels (e.g., 604a, 604b, 604c, 604d, 604e, 604f, 604g, and 604h), each of the plurality of fluid-providing channels points, in a fluid-providing direction (e.g., 605a, 605b, 605c, 605d, 605e, 605f, 605g, and 605h), toward an interior of the internal cavity (e.g., flushing cavity 603) of the flushing chamber, and each of at least some of the fluid-providing directions is different than a particular direction in which the fluid input channel (e.g., 616) providing the fluid supply input port (616-1) is configured to cause fluid to enter an interior volume within the enclosure. In some embodiments, at least a portion of the internal cavity (e.g., flushing cavity 603) of the flushing chamber is defined by at least part of a body of revolution having an axis of revolution (e.g., 612), and the particular direction in which the fluid input channel (e.g., 616) providing the fluid supply input port (e.g., 616-1) is configured to cause fluid to enter the interior volume within the enclosure (e.g., 602) has a directional component that is transversely oriented to a direction in which the axis of revolution extends. In some embodiments, the generated fluid vortex helically spins about an axis, and the particular direction in which the fluid input channel (e.g., 616) providing the fluid supply input port (616-1) is configured to cause fluid to enter the interior volume within the enclosure (e.g., 602) has a directional component that is transversely oriented to a direction in which the axis extends.

In some embodiments, method 700 includes removing fluid from the internal cavity of the flushing chamber (e.g., at least the first interior chamber 617a of the enclosure 602) during the generating the fluid vortex within the internal cavity (e.g., flushing cavity 603) of the flushing chamber. In some embodiments, method 700 may include removing fluid from the internal cavity (e.g., flushing cavity 603) of the flushing chamber (e.g., at least the first interior chamber 617a of the enclosure 602) via a fluid output port (e.g., 620-1) in fluid communication with the internal cavity (e.g., flushing cavity 603) of the flushing chamber, the fluid output port (e.g., 620-1) positioned to be encircled by the circumferential fluid flow caused by the generated fluid vortex. According to some embodiments, removing fluid from the internal cavity (e.g., flushing cavity 603) of the flushing chamber (e.g., at least the first interior chamber 617a of the enclosure 602) during the generating of the fluid vortex may cause or facilitate the fluid vortex to helically spin about an axis that extends toward a port through which the fluid is removed. The present inventors have noted enhanced flushing of undesired fluids (e.g., air) from the portion of the medical device (e.g., manipulable portion 300 or structure 308) when the fluid vortex that is generated causes a circumferential flow around the portion of the medical device positioned in the internal cavity (e.g., flushing cavity). Without being bound by any particular theory, the present inventors believe that the enhanced flushing of undesired fluids (e.g., air) arises because the circumferential flow (e.g., caused by the generated fluid vortex) that surrounds the portion of the medical device (e.g., manipulable portion 300 or structure 308) in the internal cavity (e.g., flushing cavity) causes the relatively denser flushing fluid (e.g., saline or heparinized saline) employed to flush undesired fluid (e.g., air bubbles or other gaseous bubbles) from the portion of the medical device to be centrifugally driven outward away from the portion of the medical device while the relatively less dense undesired fluid (e.g., air bubbles or other gaseous bubbles) is directed toward a region radially inward from the centrifugally driven flushing fluid or remains substantially in a region radially inward from the centrifugally driven flushing fluid. In some contexts and implementations, removing fluid from the radially inward region (e.g., via a fluid output port (e.g., 620-1)) positioned proximate an axis about which the fluid vortex spins, may effectively remove the undesired fluid. In other embodiments, the portion of the medical device may be moved away from the radially inward region after a sufficient amount of flushing has been deemed to occur. The presence of undesired fluid (e.g., air bubbles or other gaseous bubbles) may arise in some cases from (a) the introduction of the portion of the medical device (e.g., manipulable portion 300 or structure 308) into the internal cavity (e.g., flushing cavity), (b) the introduction of flushing fluid into the internal cavity (e.g., flushing cavity), or (a) and (b). Of course, the presence of undesired fluid may arise from other cases or causes.

In various embodiments, the portion of the medical device (e.g., manipulable portion 300 or structure 308) is flushed while in a deployed configuration to expose a greater amount (e.g., a greater amount of surface area) of the portion of the medical device to the flushing action and reduce the presence of crevices and other surface disruptions capable of entrapping or otherwise being a focal point for the presence of undesired fluid (e.g., air bubbles or other gaseous bubbles). In some embodiments, the portion of the medical device (e.g., manipulable portion 300 or structure 308) may be translated or reciprocated axially along the axis of the generated fluid vortex to ensure that all parts of the portion of the medical device are positioned in various regions of the fluid vortex.

In some embodiments, method 700 may include during the generating the fluid vortex within the internal cavity (e.g., flushing cavity) of the flushing chamber (e.g., enclosure 602) as per block 704, concurrently supplying fluid to the internal cavity of the flushing chamber and removing fluid from the internal cavity of the flushing chamber. In some embodiments, the fluid vortex rotates about an axis, and the method 700 includes supplying fluid to a first region of the internal cavity (e.g., flushing cavity 603) of the flushing chamber (e.g., at least the first interior chamber 617a of the enclosure 602) and removing fluid from a second region of the internal cavity of the flushing chamber, the first region of the internal cavity of the flushing chamber located further radially outward from the axis than the second region of the internal cavity of the flushing chamber. In some embodiments, the supplying fluid to the first region of the internal cavity (e.g., flushing cavity 603) of the flushing chamber (e.g., at least the first interior chamber 617a of the enclosure 602) and the removing fluid from the second region of the internal cavity of the flushing chamber occur concurrently. In some embodiments, method 700 may be employed with flushing apparatus 600 whose enclosure 602 may include a fluid output port (e.g., 620-1) fluidically coupled to the flushing cavity 603 to allow removal from the flushing cavity of at least some of the fluid provided to the flushing cavity, according to method 700. In some of these embodiments, fluid may be provided to an interior volume in the enclosure 602 via the fluid supply input port 616-1 while fluid is concurrently removed via the fluid output port 620-1. Such may allow for a continuous flow of fluid through the flushing cavity 603 thereby providing continuous flushing action. In some of these embodiments, the plurality of fluid-providing channels (e.g., 604a, 604b, 604c, 604d, 604e, 604f, 604g, and 604h) may be collectively oriented to generate a fluid vortex within the flushing cavity 603 at least in a state in which fluid is provided to the flushing cavity 603, the generated fluid vortex helically spinning about a particular axis (see, e.g., FIG. 4H, where vortex 635 is illustrated in broken line as spinning around axis 612). According to various embodiments, one of (a) the particular direction in which fluid enters an interior volume within the enclosure via a fluid input channel (e.g., 616) providing the fluid supply input port 616-1 and (b) the particular direction in which fluid is removed from the flushing cavity via the fluid output channel (e.g., 620) providing the fluid output port 620-1 is not parallel to a direction in which the particular axis extends, while the other of (a) and (b) is parallel to the direction in which the particular axis extends.

For example, according to various embodiments, flushing fluid enters an interior volume within the enclosure 602 via the fluid input channel 616 providing the fluid supply input port 616-1 along a first particular direction that is not parallel to the particular axis that the generated fluid vortex spins about, the first particular direction causing at least in part, according to some embodiments, the generation of the fluid vortex as described above in this disclosure. According to various embodiments, flushing fluid is removed from the flushing cavity 603 via the fluid output channel 620 providing the fluid output port 620-1 along a second particular direction that is parallel to the particular axis that the generated fluid vortex spins about. In some contexts and implementations, this arrangement may allow for the supplying of flushing fluid to generate, at least in part, the fluid vortex whose centrifugal action allows for the relatively less denser undesired fluid (e.g., air or other gas bubbles) to gather primarily proximate the axis where the undesired fluid is removed along with the axial removal of flushing fluid via the fluid output channel 620. According to various embodiments, the first particular direction, in which flushing fluid enters an interior volume within the enclosure 602 via the fluid input channel 616 providing the fluid supply input port 616-1, and the second particular direction, in which flushing fluid is removed from the flushing cavity 603 via the fluid output channel 620 providing the fluid output port 620-1, are non-parallel directions.

In some embodiments, a structure-receiving channel (e.g., 614) providing the structure-receiving port 614-1 and the at least one fluid-providing channel (e.g., 604a) are arranged such that a particular direction in which the distal end structure (e.g., manipulable portion 300 or structure 308) of the medical device enters the flushing cavity 603 via the structure-receiving channel 620 providing the structure-receiving port 620-1 and a particular direction in which fluid enters the flushing cavity 603 via a first fluid-providing channel of the at least one fluid-providing channel (e.g., 604a) are non-parallel directions. For example, this particular spatial arrangement may allow, the at least one fluid-providing channel (e.g., 604a) to generate a fluid vortex that easily circumferentially surrounds the distal end structure (e.g., manipulable portion 300 or structure 308) delivered into the flushing cavity 603 via the structure-receiving channel 620. In some embodiments in which the portion of the medical device is received within the internal cavity (e.g., flushing cavity 603) of the flushing chamber (e.g., at least the first interior chamber 617a of the enclosure 602) via a structure-receiving channel (e.g., 614) providing a structure-receiving port (e.g., 614-1), the structure-receiving channel (e.g., 614) may be positioned to constrain entry of the portion of the medical device into the internal cavity (e.g., flushing cavity 603) of the flushing chamber (e.g., at least the first interior chamber 617a of the enclosure 602) along a direction that does not point toward a centroid (e.g., 610) of the internal cavity of the flushing chamber. In some embodiments in which the portion of the medical device is received within the internal cavity (e.g., flushing cavity 603) of the flushing chamber (e.g., at least the first interior chamber 617a of the enclosure 602) via a structure-receiving channel (e.g., 614) providing a structure-receiving port (e.g., 614-1), the structure-receiving channel may be positioned to constrain entry of the portion of the medical device into the internal cavity (e.g., flushing cavity 603) of the flushing chamber (e.g., at least the first interior chamber 617a of the enclosure 602) along a direction that points to or generally toward (e.g., within 5, 10, 15, or 20 degrees, according to various embodiments, of) a centroid (e.g., 610) of the internal cavity of the flushing chamber (for example, as shown in FIGS. 4J and 4K).

Referring back to block 704 in FIG. 5, the fluid vortex may, in some embodiments, be generated with an apparatus other than the flushing apparatus of FIGS. 4A to 4K. For example, in some embodiments, a flushing apparatus that includes a moveable member that does not form any portion of a medical device that is to be flushed is employed by method 700 to generate a fluid vortex within the internal cavity of a flushing chamber in which a portion of the medical device is received or provided. In various embodiments, the moveable member is rotated to cause the generating the fluid vortex in the internal cavity (e.g., 603) of the flushing chamber (e.g., at least the first interior chamber 617a of the enclosure 602) as per block 704. In some embodiments, the moveable member is located within the internal cavity of the flushing chamber at least during the generating the fluid vortex within the internal cavity of the flushing chamber as per block 704. In some embodiments, the moveable member may form part of a magnetic stirrer or magnetic mixer (also known in the art as a stir bar) 609 (see, e.g., FIG. 4L) that employs a rotating magnetic field to cause the moveable member immersed in a flushing fluid to spin very quickly, thus stirring it and generating the fluid vortex. According to various embodiments, the rotating field may be created either by a set of stationary electromagnets or a rotating magnet, placed exterior of the internal cavity in which the fluid vortex is generated. Magnetic stirrers may be used in either open vessels or enclosed vessels and are sometimes preferable to mechanical stirrers since a physical rotary transmission member (e.g., accompanied by the use of various rotary seals) is not required to extend through a wall enclosing the interior cavity in which the fluid vortex is generated. Additionally, only the moveable member (e.g., stir bar) is exposed to the flushing fluid, and, as such, the sterilization burden required by the flushing apparatus for various medical device applications may be reduced. It is noted that in various embodiments, the flushing chamber is typically constructed from materials (e.g., plastics, glass) that do not appreciably affect the magnetic field employed to cause generation of the fluid vortex. In some embodiments, such as those shown in FIG. 4L, the flushing apparatus 600 may be held vertically so that the magnetic stirrer is retained in its region inside the flushing cavity 603 by gravity. In contrast, in other embodiments, such as those shown in FIGS. 4F, 4G, 4H, 4I, 4J, 4K, and 4M, the flushing apparatus 600 may be held horizontally or vertically. On the other hand, if a sufficiently strong magnetic force is utilized for the magnetic stir bar 609 in FIG. 4L, the flushing apparatus 600 may also be held horizontally.

In some embodiments where the fluid-providing channels 604 are not implemented, such as those shown in FIG. 4L and FIG. 4M, discussed below, the fluid supply input port 616-1 may be configured to cause fluid to enter directly into the flushing chamber 603 instead of, e.g., via a second interior chamber 617b, which is not implemented in some embodiments such as the embodiments of FIGS. 4L and 4M. However, although the embodiments of FIGS. 4L and 4M do not include the fluid-providing channels 604 or the second interior chamber 617b, some embodiments utilize both a moveable member, such as moveable member 609 in FIG. 4L or moveable member 607 in FIG. 4M, discussed below, as well as the fluid-providing channel 604/second interior chamber 617b configurations discussed herein.

In some embodiments, the moveable member is part of a mechanical stirrer or mechanical mixer in which the moveable member contained in the internal cavity of the flushing chamber is physically coupled to a motor to cause rotation of the moveable member to generate the fluid vortex in the internal cavity. According to various embodiments, the motor may be driven electrically, pneumatically, or hydraulically by way of non-limiting example. In some embodiments, the moveable member includes an impeller 607, as shown, for example, in FIG. 4M. In some embodiments, the moveable member is physically coupled to the motor by a transmission member. In some embodiments, the transmission member may extend through a wall of the flushing chamber that surrounds the internal cavity. Various seals may be employed to reduce fluid leakage between the transmission member and the wall of the flushing chamber, according to various embodiments.

Without limitation, method 700 may include delivering energy to a mechanical apparatus, which may include a motor (e.g., motor 608 in FIG. 4M) driven electrically, pneumatically, or hydraulically by way of non-limiting example, operatively coupled to the flushing chamber (e.g., at least the first interior chamber 617a of the enclosure 602) to facilitate the generating the fluid vortex within the internal cavity of the flushing chamber, the mechanical apparatus not forming any portion of the medical device.

Block 708 of method 700 includes removing the portion of the medical device from the internal cavity (e.g., 603) of the flushing chamber (e.g., at least the first interior chamber 617a of the enclosure 602) at least after initiation of the generating the fluid vortex within the internal cavity of the flushing chamber as per block 704. In various embodiments, the portion of the medical device is removed from the internal cavity after the portion of the medical device has been flushed or has been deemed to be flushed of an undesired fluid (e.g., air or some other gas). In some embodiments, the portion of the medical device includes a structure (e.g., manipulable portion 300 or structure 308) that is selectively moveable between a delivery configuration and a deployed configuration (examples of delivery and deployed configurations are discussed above). In some embodiments, method 700 includes moving the structure (e.g., manipulable portion 300 or structure 308) between the delivery configuration and the deployed configuration within the internal cavity of the flushing chamber at least after the initiation of the generating the fluid vortex within the internal cavity of the flushing chamber. For example, in some embodiments, the structure (e.g., manipulable portion 300 or structure 308) is moved from the delivery configuration into the deployed configuration before or during the initiation of the generating the fluid vortex within the internal cavity of the flushing chamber and may be required to be moved back to the delivery configuration at least after the initiation of the generating the fluid vortex within the internal cavity to, for example, allow removal of the portion of the medical device from the internal cavity. Accordingly, method 700 may also include in some embodiments, moving the structure (e.g., manipulable portion 300 or structure 308) between the delivery configuration and the deployed configuration within the internal cavity of the flushing chamber at least before the removing the portion of the medical device from the internal cavity of the flushing chamber.

While some of the embodiments disclosed above are suitable for the flushing of various instruments employed in cardiac mapping or ablation, the same or similar embodiments may be used for flushing various instruments used in the treatment or diagnosis or other bodily organs or any bodily lumen, bodily chamber or bodily cavity. For example, although manipulable portions 200 and 300 are often described herein as transducer-based devices, the invention is not limited to flushing of transducer-based devices, and other forms of catheter-based manipulable portions (e.g., a stent or other implant) may be utilized.

Subsets or combinations of various embodiments described above can provide further embodiments.

These and other changes may be made to the invention in light of the above-detailed description. In general, in the following claims, the terms used should not be construed to limit the invention to the specific embodiments disclosed in the specification and the claims. In this regard, the invention is not limited by the disclosure, but instead its scope is to be determined entirely by the following claims.

What is claimed is:

1. A medical device flushing apparatus comprising:
an enclosure comprising an interior surface defining at least part of a flushing cavity, the interior surface comprising a structure-receiving port configured to receive a distal end structure of a medical device, at least part of the medical device configured to be percutaneously delivered toward a bodily cavity distal end structure first; and
at least one fluid-providing channel arranged to obliquely interrupt the interior surface of the enclosure, the at least one fluid-providing channel configured to provide fluid to the flushing cavity.

2. The medical device flushing apparatus of claim 1, wherein the at least one fluid-providing channel comprises a plurality of fluid-providing channels.

3. The medical device flushing apparatus of claim 2, wherein fluid-providing channels of the plurality of fluid-providing channels are circumferentially arranged about a centroid of the flushing cavity.

4. The medical device flushing apparatus of claim 2, wherein the plurality of fluid-providing channels are aligned on a two-dimensional cross-section of the flushing cavity.

5. The medical device flushing apparatus of claim 2, wherein the flushing cavity is provided at least in part by a first interior chamber of the enclosure, and wherein the enclosure comprises a second interior chamber, the plurality of fluid-providing channels located at least in part between the first interior chamber and the second interior chamber.

6. The medical device flushing apparatus of claim 5, wherein the second interior chamber circumferentially surrounds at least part of the first interior chamber.

7. The medical device flushing apparatus of claim 6, wherein the structure-receiving port is arranged to not interrupt an internal surface of the second interior chamber.

8. The medical device flushing apparatus of claim 6, wherein a structure-receiving channel that provides the structure-receiving port does not extend through the second interior chamber.

9. The medical device flushing apparatus of claim 2, wherein:
each fluid-providing channel of the plurality of fluid-providing channels points, in a fluid-providing direction, toward an interior of the flushing cavity;
the plurality of fluid-providing channels are circumferentially arranged about an axis extending through a centroid of the flushing cavity; and
the fluid-providing directions of the plurality of fluid-providing channels point in a same rotational direction about the axis.

10. The medical device flushing apparatus of claim 2, wherein the plurality of fluid-providing channels are collectively oriented to generate a fluid vortex within the flushing cavity at least in a state in which fluid is provided to the flushing cavity.

11. The medical device flushing apparatus of claim 10, wherein each of the plurality of fluid-providing channels points, in a fluid-providing direction, toward an interior of the flushing cavity, and wherein the generated fluid vortex helically spins about an axis centrally located in the generated fluid vortex at least in the state in which fluid is provided to the flushing cavity, and wherein the fluid-providing directions of the plurality of fluid-providing channels point in a same rotational direction about the axis.

12. The medical device flushing apparatus of claim 11, wherein the enclosure comprises a fluid supply input port, wherein the enclosure fluidically couples the fluid supply input port to the plurality of fluid-providing channels of the enclosure, and wherein the fluid supply input port is spaced from the axis.

13. The medical device flushing apparatus of claim 11, wherein the structure-receiving port is spaced from the axis.

14. The medical device flushing apparatus of claim 2, wherein the enclosure comprises a fluid supply input port, wherein the enclosure fluidically couples the fluid supply input port to all of the plurality of fluid-providing channels of the enclosure, and wherein a fluid input channel providing the fluid supply input port is arranged to cause fluid to enter an interior volume within the enclosure along a particular direction that does not intersect a centroid of the flushing cavity.

15. The medical device flushing apparatus of claim 2, wherein the enclosure comprises a fluid supply input port, wherein the enclosure fluidically couples the fluid supply input port to the plurality of fluid-providing channels of the enclosure, wherein each fluid-providing channel of the plurality of fluid-providing channels points, in a fluid-providing direction, toward an interior of the flushing cavity, and wherein each of at least some of the fluid-providing directions is different than a particular direction in which a fluid input channel providing the fluid supply input port is configured to cause fluid to enter an interior volume within the enclosure.

16. The medical device flushing apparatus of claim 15, wherein the fluid-providing directions and the particular direction in which the fluid input channel providing the fluid supply input port is configured to cause fluid to enter the interior volume within the enclosure are oriented in a same rotational direction.

17. The medical device flushing apparatus of claim 15, wherein at least a portion of the flushing cavity is defined by at least part of a body of revolution having an axis of revolution, and wherein the particular direction in which the fluid input channel providing the fluid supply input port is configured to cause fluid to enter the interior volume within the enclosure has a directional component that is transversely oriented to a direction in which the axis of revolution extends.

18. The medical device flushing apparatus of claim 15, wherein the plurality of fluid-providing channels are collectively oriented to generate a fluid vortex within the flushing cavity at least in a state in which fluid is provided to the flushing cavity, the generated fluid vortex helically spinning about an axis, and wherein the particular direction in which the fluid input channel providing the fluid supply input port is configured to cause fluid to enter the interior volume within the enclosure has a directional component that is transversely oriented to a direction in which the axis extends.

19. The medical device flushing apparatus of claim 15, wherein the enclosure comprises a fluid output port fluidically coupled to the flushing cavity to allow removal from the flushing cavity of at least some of the fluid provided to the flushing cavity.

20. The medical device flushing apparatus of claim 19, wherein the fluid input channel providing the fluid supply input port and a fluid output channel providing the fluid output port are arranged such that the particular direction in which fluid enters the interior volume within the enclosure via the fluid input channel providing the fluid supply input port and a particular direction in which fluid is removed from the flushing cavity via the fluid output channel providing the fluid output port are non-parallel directions.

21. The medical device flushing apparatus of claim 20, wherein the plurality of fluid-providing channels are collectively oriented to generate a fluid vortex within the flushing cavity at least in a state in which fluid is provided to the flushing cavity, the generated fluid vortex helically spinning about an axis, and wherein one of (a) the particular direction in which fluid enters the interior volume within the enclosure via the fluid input channel providing the fluid supply input port and (b) the particular direction in which fluid is removed from the flushing cavity via the fluid output channel providing the fluid output port is not parallel to a direction in which the axis extends while the other of (a) and (b) is parallel to the direction in which the axis extends.

22. The medical device flushing apparatus of claim 1, wherein at least a portion of the flushing cavity is defined by at least part of a body of revolution having an axis of revolution.

23. The medical device flushing apparatus of claim 22, wherein the at least one fluid-providing channel comprises a plurality of fluid-providing channels, the plurality of fluid-providing channels circumferentially arranged about the axis of revolution.

24. The medical device flushing apparatus of claim 23, wherein each of the plurality of fluid-providing channels points, in a fluid-providing direction, toward an interior of the flushing cavity, the fluid-providing directions of the plurality of fluid-providing channels pointing in a same rotational direction about the axis of revolution.

25. The medical device flushing apparatus of claim 1, wherein the at least one fluid-providing channel is oriented to generate a fluid vortex within the flushing cavity at least in a state in which fluid is provided to the flushing cavity.

26. The medical device flushing apparatus of claim 1, wherein the enclosure comprises a fluid output port fluidically coupled to the flushing cavity to allow removal from the flushing cavity of at least some of the fluid provided to the flushing cavity, and wherein at least a first fluid-providing channel of the at least one fluid-providing channel and a fluid output channel providing the fluid output port are arranged such that a particular direction in which fluid enters the flushing cavity within the enclosure via the first fluid-providing channel and a particular direction in which fluid is removed from the flushing cavity via the fluid output channel providing the fluid output port are non-parallel directions.

27. The medical device flushing apparatus of claim 1, wherein a structure-receiving channel providing the structure-receiving port and the at least one fluid-providing channel are arranged such that a particular direction in which the distal end structure of the medical device enters the flushing cavity via the structure-receiving channel providing the structure-receiving port and a particular direction in which fluid enters the flushing cavity via a first fluid-providing channel of the at least one fluid-providing channel are non-parallel directions.

28. The medical device flushing apparatus of claim 1, wherein the structure-receiving port is provided by a structure-receiving channel that is positioned to constrain entry of the distal end structure of the medical device into the flushing cavity along a direction that does not point toward a centroid of the flushing cavity.

29. The medical device flushing apparatus of claim 1, wherein the distal end structure of the medical device is selectively moveable between a delivery configuration in which at least the distal end structure of the medical device is sized to be percutaneously deliverable to a bodily cavity and a deployed configuration in which at least the distal end structure of the medical device is sized to be too large to be percutaneously deliverable to the bodily cavity, and wherein the structure-receiving port is sized to receive the distal end structure of the medical device in a state in which the distal end structure of the medical device is in the delivery configuration, but not the deployed configuration.

30. The medical device flushing apparatus of claim 1, wherein the distal end structure of the medical device is selectively moveable between a delivery configuration and a deployed configuration, at least a portion of the distal end structure of the medical device having a dimension that is smaller in the delivery configuration than a corresponding dimension of the at least the portion of the distal end structure of the medical device in the deployed configuration.
